(19) United States
(12) Patent Application Publication
(10) Pub. No.: US 2014/0093661 A1
(43) Pub. Date: Apr. 3, 2014

(54) Selective Blue Light Filtered Optic

(71) Applicant: High Performance Optics, Inc., Roanoke, VA (US)

(72) Inventors: Anita Trajkovska, Roanoke, VA (US); Ronald Blum, Roanoke, VA (US); Andrew Ishak, Harve de Grace, MD (US); Sean McGinnis, Roanoke, VA (US); Michael Packard, Cincinnati, OH (US)

(73) Assignee: High Performance Optics, Inc., Roanoke, VA (US)

(21) Appl. No.: 14/042,389

(22) Filed: Sep. 30, 2013

Related U.S. Application Data
(60) Provisional application No. 61/708,668, filed on Oct. 2, 2012.
(60) Provisional application No. 61/709,414, filed on Oct. 4, 2012.
(60) Provisional application No. 61/749,481, filed on Jan. 7, 2013.
(60) Provisional application No. 61/773,911, filed on Mar. 7, 2013.
(60) Provisional application No. 61/873,327, filed on Sep. 3, 2013.
(60) Provisional application No. 61/877,280, filed on Sep. 12, 2013.

(51) Int. Cl. G02C 7/10; A61F 2/14; A61F 2/16

(52) U.S. Cl. CPC G02C 7/108; A61F 2/16; A61F 2/145
USPC 427/600; 351/159.29; 351/159.65; 427/164; 427/160; 623/6.17; 623/5.11

(57) ABSTRACT

Disclosed herein is a method that comprises providing a solution containing a dye or a dye mixture, ultrasonicating the solution to reduce the average size of aggregates of the dye or dye mixture contained in the solution, and incorporating the dye or the dye mixture in the optical path of a device that transmits light.

### CROSS-REFERENCE TO RELATED APPLICATIONS

This application claims priority from U.S. provisional patent applications Nos. 61/708,668, filed on Oct. 2, 2012, 61/709,414, filed on Oct. 4, 2012, 61/749,481, filed on Jan. 7, 2013, 61/773,911, filed on Mar. 7, 2013, 61/873,327, filed on Sep. 3, 2013, and 61/877,280, filed on Sep. 12, 2013, all of which are incorporated herein by reference in their entirety.

### TECHNICAL FIELD OF THE INVENTION

This disclosure relates generally to ophthalmic and non-ophthalmic systems. Specifically, the present disclosure describes ophthalmic and non-ophthalmic systems comprising both ultraviolet (UV) and high energy visible (HEV) light filtering, and it provides fabrication methods for manufacturing these systems.

### BACKGROUND OF THE INVENTION

Electromagnetic radiation from the sun continuously bombards the Earth's atmosphere. Light is made up of electromagnetic radiation that travels in waves. The electromagnetic spectrum includes radio waves, millimeter waves, microwaves, infrared, visible light, ultra-violet (UVA and UVB), X-rays, and gamma rays. The visible light spectrum includes the longest visible light wavelength of approximately 700 nm and the shortest of approximately 400 nm (nanometers or 10.sup.-9 meters). Blue light wavelengths fall in the approximate range of 400 nm to 500 nm. For the ultra-violet bands, UVB wavelengths are from 290 nm to 320 nm, and UVA wavelengths are from 320 nm to 400 nm. Gamma and x-rays make up the higher frequencies of this spectrum and are absorbed by the atmosphere. The wavelength spectrum of ultraviolet radiation (UVR) is 100-400 nm. Most UVR wavelengths are absorbed by the atmosphere, except where there are areas of stratospheric ozone depletion. Over the last 20 years, there has been documented depletion of the ozone layer primarily due to industrial pollution. Increased exposure to UVR has broad public health implications as an increased burden of UVR ocular and skin disease is to be expected.

The ozone layer absorbs wavelengths up to 286 nm, thus shielding living beings from exposure to radiation with the highest energy. However, we are exposed to wavelengths above 286 nm, most of which falls within the human visual spectrum (400-700 nm). The human retina responds only to the visible light portion of the electromagnetic spectrum. The shorter wavelengths pose the greatest hazard because they inversely contain more energy. Blue light has been shown to be the portion of the visible spectrum that produces the most photochemical damage to animal retinal pigment epithelium (RPE) cells. Exposure to these wavelengths has been called the blue light hazard because these wavelengths are perceived as blue by the human eye.

### SUMMARY OF THE INVENTION

Features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

In one embodiment, there is provided a method for fabricating a device that transmits light. The method comprises providing a solution containing a dye or dye mixture, and the dye or the dye mixture forms aggregates of average size less than 10 micrometers. Furthermore, the method comprises incorporating the dye or the dye mixture in the optical path of the device, and the dye or dye mixture selectively filters at least one wavelength of light within the range of 400 nm to 500 nm. Moreover, the device having the dye or dye mixture incorporated therein has an average transmission of at least 80% across the visible spectrum.

In one embodiment, the dye or dye mixture has an absorption spectrum with at least one absorption peak in the range 400 nm to 500 nm.

In one embodiment, the at least one absorption peak is in the range 400 nm to 500 nm.

In one embodiment, the at least one absorption peak has a full-width at half-max (FWHM) of less than 60 nm in the range 400 nm to 500 nm.

In one embodiment, the dye or dye mixture, when incorporated in the device's optical path, absorbs at least 5% of the at least one wavelength of light in the range 400 nm to 500 nm.

In one embodiment, the device having the dye incorporated therein has a yellowness index of 15 or less.

In one embodiment, the dye or dye mixture aggregates have an average size less than 5 micrometers.

In one embodiment, the dye or dye mixture aggregates have an average size less than 1 micrometer.

In one embodiment, providing the solution comprises ultrasonicating the solution to reduce the average size of aggregates of the dye or dye mixture contained in the solution.

In one embodiment, the ultrasonicating is performed in a controlled temperature environment.

In one embodiment, the aggregates have an average size greater than 10 micrometers prior to ultrasonicating the solution.

In one embodiment, the controlled temperature environment is set to a temperature equal or less than 50 degrees C.

In one embodiment, the incorporating comprises loading the solution in a resin to form a coating formulation.

In one embodiment, the coating formulation is subjected to further ultrasonication in a controlled temperature environment for a certain time period.

In one embodiment, the incorporating further comprises applying the coating formulation on a surface of the device.

In one embodiment, the device is an ophthalmic lens.

In one embodiment, the device is a non-ophthalmic system.

In one embodiment, the method further comprises machining a first surface of the ophthalmic lens and polishing the first surface. Furthermore, the incorporating step comprises applying a coating formulation comprising the dye or the dye mixture on the first surface to form a coating, the coating selectively inhibiting visible light in a selected range of visible wavelengths. Furthermore, the incorporating step comprises air drying or short thermal baking the coating, applying a hard scratch resistant coating on the coating,

curing the hard scratch resistant coating.

In one embodiment, the machining and the polishing provide a predetermined optical power to the ophthalmic lens.

In one embodiment, applying the coating formulation comprises determining an amount of the dye or the dye mixture, the amount corresponding to a predetermined percentage of blockage of light in the selected range.

In one embodiment, the first surface comprises a first layer which blocks ultraviolet (UV) light.

In one embodiment, a second surface of the ophthalmic lens disposed opposite to the first surface and in a plane parallel to the first surface, comprises a second layer which blocks UV light.

In one embodiment, the dye is a porphyrin or porphyrin derivative.

In one embodiment the dye is one of the group consisting of bilirubin; chlorophyll a; chlorophyll b; diprotonated-tetraphenylporphyrin; hematin; magnesium octaethylporphyrin; magnesium octaethylporphyrin (MgOEP); magnesium phthalocyanine (MgPc), PrOH; magnesium phthalocyanine (MgPc), pyridine; magnesium tetramesitylporphyrin (MgTMP); magnesium tetraphenylporphyrin (MgTPP); octaethylporphyrin; phthalocyanine (Pc); porphin; tetra-t-butylazaporphine; tetra-t-butylnaphthalocyanine; tetrakis(2,6-dichlorphenyl)porphyrin; tetrakis(o-aminophenyl)porphyrin; tetramesitylporphyrin (TMP); tetraphenylporphyrin (TPP); vitamin B12; zinc octaethylporphyrin (ZnOEP); zinc phthalocyanine (ZnPc), pyridine; zinc tetramesitylporphyrin (ZnTMP); zinc tetramesitylporphyrin radical cation; zinc tetrapheynlporphyrin (ZnTPP); perylene and derivatives thereof.

In one embodiment, the dye is tetrakis(2,6-dichlorphenyl)porphyrin (MTP).

In one embodiment, the solution includes a chlorinated solvent.

In one embodiment, the solution includes solvent having a polarity index of 3.0 or greater.

In one embodiment, the solution comprises a solvent selected from the group consisting of cyclopentanone, cyclohexanone, methyl ethyl ketone, DMSO, DMF, THF, chloroform, methylene chloride, acetonitrile, carbon tetrachloride, dichloroethane, dichloroethylene, dichloropropane, trichloroethane, trichloroethylene, tetrachloroethane, tetrachloroethylene, chlorobenzene, dichlorobenzene, and combinations thereof.

In one embodiment, the solvent of the solution is chloroform.

In one embodiment, the solvent of the solution consists essentially of chloroform.

In one embodiment, the solvent is a chlorinated solvent.

In one embodiment, the at least one wavelength of light is within the range 430 nm+/−20 nm.

In one embodiment, the at least one wavelength of light is within the range 430 nm+/−30 nm.

In one embodiment, the at least one wavelength of light is within the range 420 nm+/−20 nm.

In one embodiment, the coating is a primer coating.

In one embodiment, the method further comprises incorporating at least one of a UV-blocking component and an IR-blocking component in the optical path of the device.

In one embodiment, the method further comprises incorporating at least one of a UV-blocking component and an IR-blocking component in the optical path of the device.

In one embodiment, the device selectively filters the at least one wavelength in the range of 400 nm to 500 nm using at least one of a reflective coating and a multi-layer interference coating.

In one embodiment, the dye or dye mixture, when incorporated in the device's optical path, absorbs 5-50% of light in the range 400 nm to 500 nm.

In one embodiment, the dye or dye mixture, when incorporated in the device's optical path, absorbs 20-40% of light in the range 400 nm to 500 nm.

In one embodiment, the device blocks 5-50% of light in the range 400 nm to 500 nm.

In one embodiment, the device blocks 20-40% of light in the range 400 nm to 500 nm.

In one embodiment, the controlled temperature environment is set at a temperature equal to or less than 50 degrees C. and the time period is between 1 hour and 5 hours.

In one embodiment, the dye or dye mixture has a Soret peak within the range 400 nm to 500 nm.

In one embodiment, the at least one absorption peak has a full-width at half-max (FWHM) of less than 40 nm in the range 400 nm to 500 nm.

In one embodiment, the at least one wavelength is 430 nm.

In one embodiment, The method of claim 1, wherein the dye or dye mixture, when incorporated in the device's optical path, absorbs 5-50% of light in the range 410 nm to 450 nm.

In one embodiment, the dye or dye mixture, when incorporated in the device's optical path, absorbs 20-40% of light in the range 410 nm to 450 nm.

In one embodiment, the device blocks 5-50% of light in the range 410 nm to 450 nm.

In one embodiment, the device blocks 20-40% of light in the range 410 nm to 450 nm.

In one embodiment, the dye or dye mixture, when incorporated in the device's optical path, absorbs 5-50% of light in the range 400 nm to 460 nm.

In one embodiment, the dye or dye mixture, when incorporated in the device's optical path, absorbs 20-40% of light in the range 400 nm to 460 nm.

In one embodiment, the device blocks 5-50% of light in the range 400 nm to 460 nm.

In one embodiment, the device blocks 20-40% of light in the range 400 nm to 460 nm.

In one embodiment, the dye or dye mixture, when incorporated in the device's optical path, absorbs 5-50% of light in the range 400 nm to 440 nm.

In one embodiment, the dye or dye mixture, when incorporated in the device's optical path, absorbs 20-40% of light in the range 400 nm to 440 nm.

In one embodiment, the device blocks 5-50% of light in the range 400 nm to 440 nm.

In one embodiment, the device blocks 20-40% of light in the range 400 nm to 440 nm.

In one embodiment, the haze level of the device having incorporated therein the dye or dye mixture therein is less than 0.6%.

In one embodiment, there is provided an ophthalmic system which comprises an ophthalmic lens selected from the group consisting of a spectacle lens, contact lens, intra-ocular lens, corneal inlay, corneal onlay, corneal graft, and corneal tissue, and a selective light wavelength filter that blocks 5-50% of light having a wavelength in the range between 400-500 nm and transmits at least 80% of light across the visible spectrum. Further, the selective wavelength filter comprises a dye or a dye mixture having average aggregate size of less than 1 micrometer.

In one embodiment, the system exhibits a yellowness index of no more than 15.

In one embodiment, the system has a haze level of less than 0.6%.

In one embodiment, the range is 400-460 nm.

In one embodiment, there is provided a method comprising providing a solution containing a dye or a dye mixture, ultrasonicating the solution to reduce the average size of aggregates of the dye or dye mixture contained in the solution, and incorporating the dye or the dye mixture in the optical path of a device that transmit light.

in one embodiment, there is provided an ophthalmic system prepared by a process comprising providing a solution containing a dye or dye mixture, the dye or the dye mixture forming aggregates of average size less than 10 micrometers, incorporating the dye or the dye mixture in the optical path of the ophthalmic lens, and the dye or dye mixture selectively filters at least one wavelength of light within the range of 400 nm to 500 nm. Further, the system having the dye or dye mixture incorporated therein has an average transmission of at least 80% across the visible spectrum.

In one embodiment, the ophthalmic system comprises an ophthalmic lens, the ophthalmic lens selected from the group consisting of a spectacle lens, contact lens, intra-ocular lens, corneal inlay, corneal onlay, corneal graft, and corneal tissue. Further, the ophthalmic system comprises a selective light wavelength filter that blocks 5-50% of light having a wavelength in the range of 400-500 urn and transmits at least 80% of light across the visible spectrum, the selective wavelength filter comprising the dye or dye mixture

In one embodiment, the system exhibits a yellowness index of no more than 15.

In one embodiment, the haze level of the ophthalmic system is less than 0.6%.

### BRIEF DESCRIPTION OF THE DRAWINGS/FIGURES

Embodiments of the invention will now be described, by way of example only, with reference to the accompanying schematic drawings. The accompanying drawings, which are incorporated herein and form part of the specification, illustrate the present disclosure and further serve to explain the principles disclosed.

FIG. 1 illustrates the percentage of cell death reduction as a function of selective blue light blockage.

FIG. 2 shows a method of fabricating a device on a CR-39 substrate, according to an embodiment.

FIG. 3 shows a method of fabricating a device on a polycarbonate substrate, according to an embodiment.

FIG. 4 shows a method of fabricating a device on an MR-8 substrate, according to an embodiment.

FIG. 5 shows a method of fabricating a device on an MR-8 substrate equipped with UV-blocking, according to an embodiment.

FIG. 6 shows a method of fabricating a device on an MR-7 substrate, according to an embodiment.

FIG. 7 shows a method of fabricating a device on an MR-10 substrate, according to an embodiment.

FIG. 8 shows the yellowness index of a device as a function of selective blue light blockage percentage for a device fabricated on a CR-9 substrate, according to an embodiment.

FIG. 9 shows the yellowness index of a device as a function of selective blue light blockage percentage for a device fabricated on a polycarbonate substrate, according to an embodiment.

FIG. 10 shows the yellowness index of a device as a function of selective blue light blockage percentage for a device fabricated on an MR-8 substrate, according to an embodiment.

FIG. 11 shows the yellowness index of a device as a function of selective blue light blockage percentage for a device fabricated on an MR-7 substrate, according to an embodiment.

FIG. 12 shows the yellowness index of a device as a function of selective blue light blockage percentage for a device fabricated on an MR-10, according to an embodiment.

FIG. 13 shows the yellowness of index as a function of selective blue light blockage percentage for several devices fabricated on different substrates, according to an embodiment.

FIG. 14 shows a method of fabricating a device on a CR-39 substrate and of providing UV-blocking on front and back sides of the substrate, according to an embodiment.

FIG. 15 shows a method of fabricating a device on an MR-8 substrate and of providing UV-blocking on front and back sides of the substrate, according to an embodiment.

FIG. 16A shows an ophthalmic system comprising a CR-9 substrate, according to an embodiment.

FIG. 16B shows an ophthalmic system comprising a CR-39 substrate and UV-blocking layers on its outermost surfaces, according to an embodiment.

FIG. 16C shows an ophthalmic system comprising a CR-39 substrate and UV-blocking layers and anti-reflective (AR) coatings on the outermost layers, according to an embodiment.

FIG. 17 shows an ophthalmic system comprising a substrate which has intrinsic UV-blocking capability, according to an embodiment.

The features and advantages of the present invention will become more apparent from the detailed description set forth below when taken in conjunction with the drawings, in which like reference characters identify corresponding elements throughout. Additionally, generally, the left-most digit(s) of a reference number identifies the drawing in which the reference number first appears.

### DETAILED DESCRIPTION OF THE INVENTION

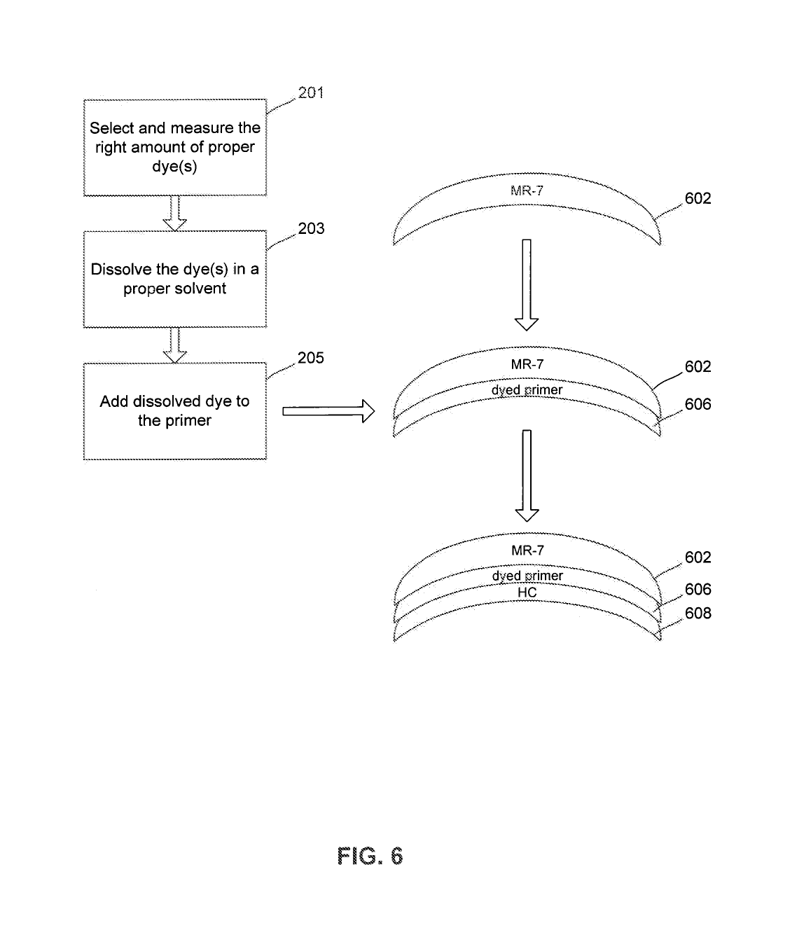

Cataracts and macular degeneration are believed to result from photochemical damage to the intraocular lens and retina, respectively. Blue light exposure has also been shown to accelerate proliferation of uveal melanoma cells. The most energetic photons in the visible spectrum have wavelengths between 380 and 500 nm and are perceived as violet or blue. The wavelength dependence of phototoxicity summed over all mechanisms is often represented as an action spectrum, such as is described in Mainster and Sparrow, “How Much Blue Light Should an IOL Transmit?” Br. J. Ophthalmol., 2003, v. 87, pp. 1523-29 and FIG. 6. In eyes without an intraocular lens (aphakic eyes), light with wavelengths shorter than 400 nm can cause damage. In phakic eyes, this light is absorbed by the intraocular lens and therefore does not contribute to retinal phototoxicity; however it can cause optical degradation of the lens or cataracts.

The pupil of the eye responds to the photopic retinal illuminance, in trolands (a unit of conventional retinal illuminance; a method for correcting photometric measurements of luminance values impinging on the human eye by scaling them by the effective pupil size), which is the product of the incident flux with the wavelength-dependent sensitivity of the retina and the projected area of the pupil. This sensitivity is described in Wyszecki and Stiles, Color Science: Concepts and Methods, Quantitative Data and Formulae (Wiley: N.Y.) 1982, esp. pages 102-107.

Current research strongly supports the premise that short wavelength visible light (blue light) having a wavelength of approximately 400-500 nm could be a contributing cause of AMD (age related macular degeneration). It is believed that the highest level of blue light retinal damage occurs in a region around 430 nm, such as 400-460 nm. Research further suggests that blue light worsens other causative factors in AMD, such as heredity, tobacco smoke, and excessive alcohol consumption.

The human retina includes multiple layers. These layers listed in order from the first exposed to any light entering the eye to the deepest include: 1) Nerve Fiber Layer 2) Ganglion Cells 3) Inner Plexiform Layer 4) Bipolar and Horizontal Cells 5) Outer Plexiform Layer 6) Photoreceptors (Rods and Cones) 7) Retinal Pigment Epithelium (RPE) 8) Bruch's Membrane 9) Choroid.

When light is absorbed by the eye's photoreceptor cells, (rods and cones) the cells bleach and become unreceptive until they recover. This recovery process is a metabolic process and is called the “visual cycle.” Absorption of blue light has been shown to reverse this process prematurely. This premature reversal increases the risk of oxidative damage and is believed to lead to the buildup of the pigment lipofuscin in the retina. This build up occurs in the retinal pigment epithelium (RPE) layer. It is believed that aggregates of extra-cellular materials called drusen are formed due to the excessive amounts of lipofuscin.

Current research indicates that over the course of one's life, beginning with that of an infant, metabolic waste byproducts accumulate within the pigment epithelium layer of the retina, due to light interactions with the retina. This metabolic waste product is characterized by certain fluorophores, one of the most prominent being lipofuscin constituent A2E. In vitro studies by Sparrow indicate that lipofuscin chromophore A2E found within the RPE is maximally excited by 430 nm light. It is theorized that a tipping point is reached when a combination of a build-up of this metabolic waste (specifically the lipofuscin fluorophore) has achieved a certain level of accumulation, the human body's physiological ability to metabolize within the retina certain of this waste has diminished as one reaches a certain age threshold, and a blue light stimulus of the proper wavelength causes drusen to be formed in the RPE layer. It is believed that the drusen then farther interfere with the normal physiology/metabolic activity which allows for the proper nutrients to get to the photoreceptors thus contributing to age-related macular degeneration (AMD). AMD is the leading cause of irreversible severe visual acuity loss in the United States and Western World. The burden of AMD is expected to increase dramatically in the next 20 years because of the projected shift in population and the overall increase in the number of ageing individuals.

Drusen hinder or block the RPE layer from providing the proper nutrients to the photoreceptors, which leads to damage or even death of these cells. To further complicate this process, it appears that when lipofuscin absorbs blue light in high quantities it becomes toxic, causing further damage and/or death of the RPE cells. It is believed that the lipofuscin constituent A2E is at least partly responsible for the short wavelength sensitivity of RPE cells. A2E has been shown to be maximally excited by blue light; the photochemical events resulting from such excitation can lead to cell death. See, for example, Janet R. Sparrow et al., “Blue light-absorbing intraocular lens and retinal pigment epithelium protection in vitro,” J. Cataract Refract. Surg. 2004, vol. 30, pp. 873-78. A reduction in short-wavelength transmission in an ophthalmic system may be useful in reducing cell death due to photoelectric effects in the eye, such as excitation of A2E, a lipofuscin fluorophore.

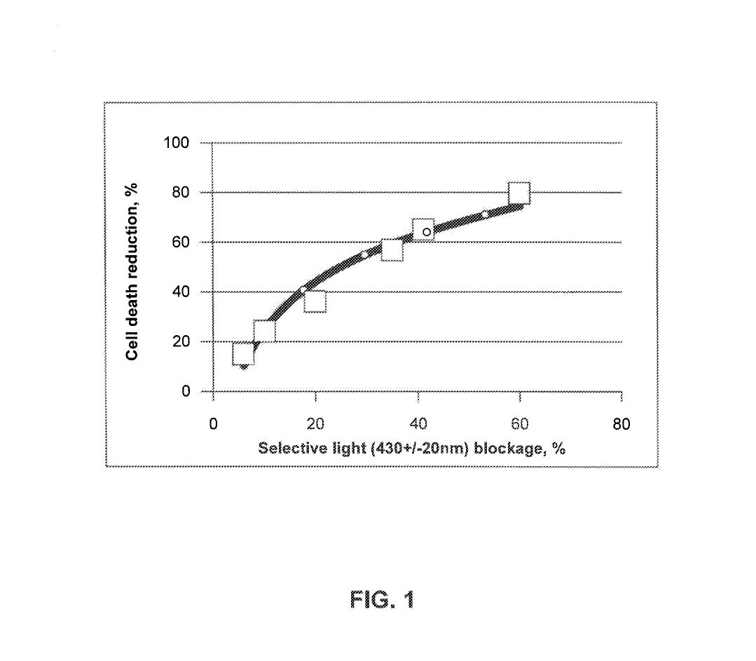

It has been shown that reducing incident light at 430+/−30 nm by about 50% can reduce cell death by about 80%. See, for example, Janet R. Sparrow et al., “Blue light-absorbing intraocular lens and retinal pigment epithelium protection in vitro,” J. Cataract Refract. Surg. 2004, vol. 30, pp. 873-78, the disclosure of which is incorporated by reference in its entirety. It is further believed that reducing the amount of blue light, such as light in the 430-460 nm range, by as little as 5% may similarly reduce cell death and/or degeneration, and therefore prevent or reduce the adverse effects of conditions such as atrophic age-related macular degeneration. FIG. 1 shows the percentage of cell death reduction as a function of selective blue light (430+/−20 nm) blockage percentage.

Further laboratory evidence by Sparrow at Columbia University for High Performance Optics has shown that concentrations of blue light filtering dyes with levels as low as 1.0 ppm and 1.9 ppm can provide retinal benefit in a mostly colorless system, “Light Filtering in Retinal Pigment Epithelial Cell Culture Model” Optometry and Vision Science 88; 6 (2011): 1-7, is referenced in its entirety. As shown in FIGS. 51 and 52 of the Sparrow report it is possible to vary the concentration of the filter system to a level of 1.0 ppm or greater to a level of about 35 ppm as exampled with perylene dye. Any concentration level between about 1.0 ppm or greater to about 35 ppm can enable the invention. Other dyes that exhibit similar blue light blocking function could also be used with similar variable dye concentration levels.

The following table demonstrates RPE cell death reduction as light blockage percentages increase with the porphyrin dye, MTP.

TABLE 1

| cell death | Light blockage, % |
|---|---|
| reduction % | 410-450 nm |
| 15 | 6 |
| 24 | 10 |
| 36 | 20 |
| 57 | 35 |
| 65 | 41 |
| 80 | 60 |

From a theoretical perspective, the following appears to take place: 1) Waste buildup occurs within the pigment epithelial level starting from infancy throughout life. 2) Retinal metabolic activity and ability to deal with this waste typically diminish with age. 3) The macula pigment typically decreases as one ages, thus filtering out less blue light. 4) Blue light causes the lipofuscin to become toxic. The resulting toxicity damages pigment epithelial cells.

The lighting and vision care industries have standards as to human vision exposure to UVA and UVB radiation. No such standard is in place with regard to blue light. For example, in the common fluorescent tubes available today, the glass envelope mostly blocks ultra-violet light but blue light is transmitted with little attenuation. In some cases, the envelope is designed to have enhanced transmission in the blue region of the spectrum. Such artificial sources of light hazard may also cause eye damage. There is also mounting concern that exposure to LED lights may impact retinal integrity.

Laboratory evidence by Sparrow at Columbia University has shown that if about 50% of the blue light within the wavelength range of 430+/−30 nm is blocked, RPE cell death caused by the blue light may be reduced by up to 80%. External eyewear such as sunglasses, spectacles, goggles, and contact lenses that block blue light in an attempt to improve eye health are disclosed, for example, in U.S. Pat. No. 6,955,430 to Pratt. Other ophthalmic devices whose object is to protect the retina from this phototoxic light include intraocular and contact lenses. These ophthalmic devices are positioned in the optical path between environmental light and the retina and generally contain or are coated with dyes that selectively absorb blue and violet light.

Other lenses are known that attempt to decrease chromatic aberration by blocking blue light. Chromatic aberration is caused by optical dispersion of ocular media including the cornea, intraocular lens, aqueous humour, and vitreous humour. This dispersion focuses blue light at a different image plane than light at longer wavelengths, leading to defocus of the full color image. Conventional blue blocking lenses are described in U.S. Pat. No. 6,158,862 to Patel et al., U.S. Pat. No. 5,662,707 to Jinkerson, U.S. Pat. No. 5,400,175 to Johansen, and U.S. Pat. No. 4,878,748 to Johansen.

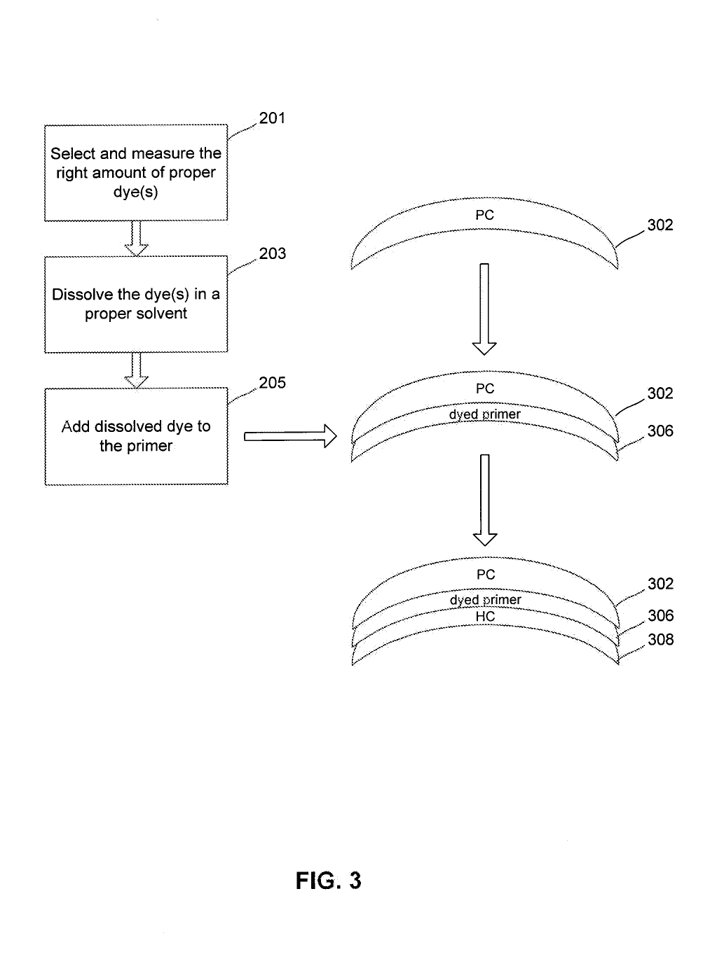

Conventional methods for reducing blue light exposure of ocular media typically completely occlude light below a threshold wavelength, while also reducing light exposure at longer wavelengths. For example, the lenses described in U.S. Pat. No. 6,955,430 to Pratt transmits less than 40% of the incident light at wavelengths as long as 650 nm, as shown in FIG. 6 of Pratt '430. The blue-light blocking lens disclosed by Johansen and Diffendaffer in U.S. Pat. No. 5,400,175 similarly attenuates light by more than 60% throughout the visible spectrum, as illustrated in FIG. 3 of the '175 patent.

Balancing the range and amount of blocked blue light may be difficult, as blocking and/or inhibiting blue light affects color balance, color vision if one looks through the optical device, and the color in which the optical device is perceived. For example, shooting glasses appear bright yellow and block blue light. The shooting glasses often cause certain colors to become more apparent when one is looking into a blue sky, allowing for the shooter to see the object being targeted sooner and more accurately. While this works well for shooting glasses, it would be unacceptable for many ophthalmic applications. In particular, such ophthalmic systems may be cosmetically unappealing because of a yellow or amber tint that is produced in lenses by blue blocking. More specifically, one common technique for blue blocking involves tinting or dyeing lenses with a blue blocking tint, such as BPI Filter Vision 450 or BPI Diamond Dye 500. The tinting may be accomplished, for example, by immersing the lens in a heated tint pot containing a blue blocking dye solution for some predetermined period of time. Typically, the solution has a yellow or amber color and thus imparts a yellow or amber tint to the lens. To many people, the appearance of this yellow or amber tint may be undesirable cosmetically. Moreover, the tint may interfere with the normal color perception of a lens user, making it difficult, for example, to correctly perceive the color of a traffic light or sign.

Efforts have been made to compensate for the yellowing effect of conventional blue blocking filters. For example, blue blocking lenses have been treated with additional dyes, such as blue, red or green dyes, to offset the yellowing effect. The treatment causes the additional dyes to become intermixed with the original blue blocking dyes. However, while this technique may reduce yellow in a blue blocked lens, intermixing of the dyes may reduce the effectiveness of the blue blocking by allowing more of the blue light spectrum through. Moreover, these conventional techniques undesirably reduce the overall transmission of light wavelengths other than blue light wavelengths. This unwanted reduction may in turn result in reduced visual acuity for a lens user.

It has been found that conventional blue-blocking reduces visible transmission, which in turn stimulates dilation of the pupil. Dilation of the pupil increases the flux of light to the internal eye structures including the intraocular lens and retina. Since the radiant flux to these structures increases as the square of the pupil diameter, a lens that blocks half of the blue light but, with reduced visible transmission, relaxes the pupil from 2 mm to 3 mm diameter will actually increase the dose of blue photons to the retina by 12.5%. Protection of the retina from phototoxic light depends on the amount of this light that impinges on the retina, which depends on the transmission properties of the ocular media and also on the dynamic aperture of the pupil. Previous work to date has been silent on the contribution of the pupil to prophylaxis of phototoxic blue light.

Another problem with conventional blue-blocking is that it can degrade night vision. Blue light is more important for low-light level or scotopic vision than for bright light or photopic vision, a result which is expressed quantitatively in the luminous sensitivity spectra for scotopic and photopic vision. Photochemical and oxidative reactions cause the absorption of 400 to 450 nm light by intraocular lens tissue to increase naturally with age. Although the number of rod photoreceptors on the retina that are responsible for low-light vision also decreases with age, the increased absorption by the intraocular lens is important to degrading night vision. For example, scotopic visual sensitivity is reduced by 33% in a 53 yew-old lens and 75% in a 75 year-old lens. The tension between retinal protection and scotopic sensitivity is further described in Mainster and Sparrow, “How Much Light Should and IOL Transmit?” Br. J. Ophthalmol., 2003, v. 87, pp. 1523-29.

Conventional approaches to blue blocking also may include cutoff or high-pass filters to reduce the transmission below a specified blue or violet wavelength to zero. For example, all light below a threshold wavelength may be blocked completely or almost completely. For example, U.S. Pub. Patent Application No. 2005/0243272 to Mainster and Mainster, “Intraocular Lenses Should Block UV Radiation and Violet but not Blue Light,” Arch. Ophthal., v. 123, p. 550 (2005) describe the blocking of all light below a threshold wavelength between 400 and 450 nm. Such blocking may be undesirable, since as the edge of the long-pass filter is shifted to longer wavelengths, dilation of the pupil acts to increase the total flux. As previously described, this can degrade scotopic sensitivity and increase color distortion.

Recently there has been debate in the field of intraocular lenses (IOLs) regarding appropriate UV and blue light blocking while maintaining acceptable photopic vision, scotopic vision, color vision, and circadian rhythms.

In view of the foregoing, there is a pressing need for an ophthalmic or non-ophthalmic system that can provide one or more of the following: 1) Blue blocking with an acceptable level of blue light protection 2) Acceptable color cosmetics, i.e., it is perceived as mostly color neutral by someone observing the ophthalmic system when worn by a wearer. 3) Acceptable color perception for a user. In particular, there is a need for an ophthalmic system that will not impair the wearer's color vision and further that reflections from the back surface of the system into the eye of the wearer be at a level of not being objectionable to the wearer. 4) Acceptable level of light transmission for wavelengths other than blue light wavelengths. In particular, there is a need for an ophthalmic system that allows for selective blockage of wavelengths of blue light while at the same time transmitting in excess of 80% of visible light. 5) Acceptable photopic vision, scotopic vision, color vision, and/or circadian rhythms.

In order to provide this optimal ophthalmic system it is desirable to include standardized Yellowness Index ranges, whereby the upper end of said range closely borders a cosmetically unacceptable yellow color. The coating may be applied to any ophthalmic system, by way of example only: an eyeglass lens, a sunglass lens, a contact lens, intra-ocular lens, corneal inlay, corneal onlay, corneal graft, electro-active ophthalmic system or any other type of lens or non-ophthalmic system as long as the Yellowness Index is 15.0 or less.

It is also known that by reducing blue light dose to the retina one can increase contrast sensitivity by reducing by way of example only the Rayleigh effect. Therefore, embodiments of the invention have a two-fold function to reduce RPE cell death and/or increase contrast sensitivity.

In one embodiment of the invention, a contact lens comprises a dye and formulated such that it will not leach out of the contact lens material. The dye is further formulated such that it provides a tint having a yellow cast. This yellow cast allows for the contact lens to have what is known as a handling tint for the wearer. This filtering provides retinal protection and enhanced contrast sensitivity without compromising in any meaningful way one's photopic vision, scotopic vision, color vision, or circadian rhythms.

In the case the embodiment of the invention is a contact lens the dye or pigment can be imparted into the contact lens by way of example only, by imbibing, so that it is located within a central 10, 11, 12, 13, or 14 mm diameter or less circle of the contact lens, or within 2-9 mm diameter of the center of the contact lens coinciding with the pupil of the wearer. In this embodiment the dye or pigment concentration which provides selective light wavelength filtering is increased to a level that provides the wearer with an increase in contrast sensitivity (as oppose to without wearing the contact lens) and without compromising in any meaningful way (one or more, or all of) the wearer's photopic vision, scotopic vision, color vision, or circadian rhythms. Further: rings, layers, or zones of filtering may optionally be included.

Preferably, an increase in contrast sensitivity is demonstrated by an increase in the user's Functional Acuity Contrast Test (FACT) score of at least about 0.1, 0.25, 0.3, 0.5, 0.7, 1, 1.25, 1.4, or 1.5. With respect to the wearer's photopic vision, scotopic vision, color vision, and/or circadian rhythms, the ophthalmic system preferably maintains one or all of these characteristics to within 15%, 10%, 5%, or 1% of the characteristic levels without the ophthalmic system.

In another embodiment that utilizes a contact lens the dye or pigment is provided that causes a yellowish tint that it is located over the central 2-9 mm diameter of the contact lens and wherein a second color tint is added peripherally to that of the central tint. In this embodiment the dye concentration which provides selective light wavelength filtering is increased to a level that provides the wearer very good contrast sensitivity and once again without compromising in any meaningful way (one or more, or all of) the wearer's photopic vision, scotopic vision, color vision, or circadian rhythms.

In still another embodiment that utilizes a contact lens the dye or pigment is provided such that it is located over the full diameter of the contact lens from approximately one edge to the other edge. In this embodiment the dye concentration which provides selective light wavelength filtering is increased to a level that provides the wearer very good contrast sensitivity and once again without compromising in any meaningful way (one or more, or all of) the wearer's photopic vision, scotopic vision, color vision, or circadian rhythms.

When various embodiments are used in or on human or animal tissue the dye is formulated in such a way to chemically bond to the inlay substrate material thus ensuring it will not leach out in the surrounding corneal tissue. Methods for providing a chemical hook that allow for this bonding are well known within the chemical and polymer industries.

In still another embodiment an intraocular lens includes a selective light wavelength filter that has a yellowish tint, and that further provides the wearer improved contrast sensitivity without compromising in any meaningful way (one or more, or all of) the wearer's photopic vision, scotopic vision, color vision, or circadian rhythms. When the selective filter is utilized on or within an intraocular lens it is possible to increase the level of the dye or pigment beyond that of a spectacle lens as the cosmetics of the intraocular lens are invisible to someone looking at the wearer. This allows for the ability to increase the concentration of the dye or pigment and provides even higher levels of improved contrast sensitivity and/or retinal protection without compromising in any meaningful way (one or more, or all of) the wearer's photopic vision, scotopic vision, color vision, or circadian rhythms.

In still another embodiment of the invention, a spectacle lens includes a selective light wave length filter comprising a dye wherein the dye's formulation provides a spectacle lens that has a mostly colorless appearance. And furthermore that provides the wearer with improved contrast sensitivity without compromising in any meaningful way (one or more, or all of) the wearer's photopic vision, scotopic vision, color vision, or circadian rhythm.

Other embodiments of the invention include a wide variation in how the selective filter can be added to any system in varying concentrations and/or zones and/or rings and/or layers. For example, in an eyeglass lens the select filter does not necessarily need to be uniform throughout the entire system or in any fixed concentration. An ophthalmic lens could have one or more zones and/or rings and/or layers of varying filter concentration or any combination or combinations thereof.

In order to cost effectively incorporate selective visible light filtering in either an ophthalmic or non-ophthalmic system a coating that includes the filtering system is the basis of the invention. By way of example only, the coating described can be incorporated into one or more than one: primer coats, scratch coats, anti-reflective coats, hydrophobic coats or other coatings known in the ophthalmic or non-ophthalmic industry or any combination or combinations thereof.

A coating is provided specifically adapted through the use of a dye to selectively inhibit transmission of visible light between 450+/−50 nm or other blue light wavelength ranges wherein the yellowness index is 15.0 or less. It is further provided that the selective filter can also be included in a broad blocking visible light filter system, whereby said system either improves contrast and/or retinal protection. It is also provided that the selective filter may have one or more peaks within 450+/−50 nm or other blue light wavelength ranges as long as the yellowness index is 15.0 or less. Further a broad blocking dye along with one or more selective wavelength peaks within 450+/−50 nm or other blue light wavelength ranges may optionally be included as long as the yellowness index is 15.0 or less.

It is further provided a coating as described above that inhibits at least 5%, preferably at least 10%, or 20%, or maximally 30% of light having a wavelength of 450+/−50 nm or 430+/−30 nm or 420+/−20 nm or 430+/−20 nm.

Also, provided is a coating as described above that selectively inhibits transmission of at least two different ranges or peaks of wavelengths selected from the range of 450+/−50 nm or 430+/−30 nm or 420+/−20 nm or 430+/−20 nm. Further, optionally a broad blocking dye within 450+/−50 nm may also be added as long as the yellowness index is 15.0 or less.

A coating as described above is provided that blocks at least 5%, preferably at least 10%, or 20%, or maximally 30% of light having a wavelength of X1.+−.15 nm, and at least 5% or 10% or 20% or maximally 30% of the light having a wavelength of X2.+−0.15 nm, where X1 is a wavelength in the range of 415-485 nm and X2 is a wavelength different from X1 and in the range of 415-485 nm.

A coating as described above is provided that transmits at least 80% of all light wavelengths in the range of 400-500 nm, except light wavelengths at X1.+−.15 nm and X2.+−.15 nm, where X1 is a wavelength in the range of 415-485 nm and X2 is a wavelength different from X1 and in the range of 415-485 nm.

A coating as described above is provided whereby the coating is applied to a spectacle lens or other type of lens and has a yellowness index not more than 15.0 or preferably not more than 12.5 or preferably not more than 10.0 or preferably not more than 8.0 or most preferably not more than 7.0.

A coating as described above is provided where the coating is applied to spectacle lens and blocks at least 5%, preferably at least 10% of light having a wavelength of 450+/−50 nm, or other blue light wavelength ranges while having an average transmission of at least 80%, or at least 85%, or most preferably at least 90% across the visible spectrum.

A coating as described above is provided where the coating is applied to a spectacle lens and selectively inhibits visible light between 430+/−30 nm. This spectacle lens may also block at least 5%, preferably at least 10%, more preferably at least 15%, more preferably at least 20%, of light having a wavelength of 430+/−30 nm, while having an average transmission of at least 80%, or at least 85%, or most preferably at least 90% across the visible spectrum.

A coating as described above is provided whereby the coating is applied to a spectacle lens and selectively inhibits visible light between 430+/−20 nm. This spectacle lens may also block at least 5%, at least 10%, or 15%, or maximally 20% within the 420+/−20 nm range, while having an average transmission of at least 80%, or at least 85%, or most preferably at least 90% across the visible spectrum.

A coating as described above is also provided whereby the coating is applied to a spectacle lens, sunglass lens, contact lens, intra-ocular lens, corneal inlay, corneal onlay, corneal graft, corneal tissue, electro-active ophthalmic system or a non-ophthalmic system and selectively inhibits visible light between 430+/−20 nm, whereby the coating blocks a maximum of 30% of light within the 430+/−20 nm range with a yellowness index of 15.0 or less. In one embodiment, the lens made with the process discussed above, with respect to embodiments of the invention, can have yellowness index (YI) of 15.0 or less. In other embodiments of the invention a YI of 10.0 or less, or 9.0 or less, or 8.0 or less, or 7.0 or less, or 6.0 or less, or 5.0 or less, or 4.0 or less, or 3.0 or less is preferred to reduce blue light dose to the retina and allow best possible cosmetics of the intended application. The YI varies based on the specific filter application

A coating as described above is provided that may also selectively inhibit transmission of light within the UV wavelength range, and optionally in the IR range.

A coating as described can be combined with one or more: primer, scratch, hydrophobic, anti-reflective, UV, IR or any other type of additional component to an ophthalmic or non-ophthalmic system.

A coating as described above is provided whereby the coating contains a dye that causes the lens to selectively inhibit transmission of visible light between 450+/−50 nm or 430+/−30 nm or 420+/−20 nm.

The dye may be selected from the following: bilirubin; chlorophyll a; chlorophyll b; diprotonated-tetraphenylporphyrin; hematin; magnesium octaethylporphyrin; magnesium octaethylporphyrin (MgOEP); magnesium phthalocyanine (MgPc), PrOH; magnesium phthalocyanine (MgPc), pyridine; magnesium tetramesitylporphyrin (MgTMP); magnesium tetraphenylporphyrin (MgTPP); octaethylporphyrin; phthalocyanine (Pc); porphin; tetra-t-butylazaporphine; tetra-t-butylnaphthalocyanine; tetrakis(2,6-dichlorphenyl)porphyrin; tetrakis(o-aminophenyl) porphyrin; tetramesitylporphyrin (TMP); tetraphenylporphyrin (TPP); vitamin B12; zinc octaethylporphyrin (ZnOEP); zinc phthalocyanine (ZnPc), pyridine; zinc tetramesitylporphyrin (ZnTMP); zinc tetramesitylporphyrin radical cation; zinc tetrapheynlporphyrin (ZnTPP); perylene and derivatives thereof.

A coating as described above is provided where the lens contains a dye where the dye is perylene or magnesium tetramesitylporphyrin (MgTMP) or magnesium tetraphenylporphyrin (MgTPP) or tetrakis(2,6-dichlorophenyl)porphyrin or meso-Tetra(o-dichlorophenyl)porphine or MTP.

A non-ophthalmic system is provided specifically adapted to include the coating that selectively inhibits transmission of visible light between 450+/−50 nm or 460+/−30 nm or 420+/−20 nm, or 430+/−20 nm wherein the has a yellowness index not more than 15.0.

A non-ophthalmic system as described above adapted to include the coating that provides blocking of at least 5%, preferably at least 10%, more preferably at least 20%, or maximally 30% of light having a wavelength of 450+/−30 nm or 430+/−30 nm or 420+/−20 nm, or 430+/−20 nm while having an average light transmission of at least 80% or at least 85%, or most preferably at least 90% across the visible spectrum.

A non-ophthalmic system as described above is provided where the yellowness index is not more than 15.0. The non-ophthalmic optic made with the invention can have yellowness index (YI) of 15.0 or less. In other embodiments of the invention a YI of 10.0 or less, or 9.0 or less, or 8.0 or less, or 7.0 or less, or 6.0 or less, or 5.0 or less, or 4.0 or less, or 3.0 or less is preferred to reduce blue light dose to the retina and allow best possible cosmetics of the intended application. The YI varies based on the specific filter application.

Embodiments of the present invention include a coating designed to selectively inhibit high energy visible light in the 450+/−50 nm range or 430+/−30 nm range or 420+/−20 nm range, or 430+/−20 nm range, or other blue light wavelength ranges, whereby the system can be incorporated in an ophthalmic or non-ophthalmic system, wherein the system has an average transmission of at least 80%, or at least 85%, or most preferably at least 90% across the visible spectrum, wherein the Yellowness Index is 15.0 or less.

Selective filtering allows for the blocking of harmful wavelengths of light, at high overall light transmission levels, without color shift. A “color shift” as used herein refers to the amount by which the CIE coordinates of a reference light change after transmission and/or reflection of the ophthalmic system. It also may be useful to characterize a system by the color shift causes by the system due to the differences in various types of light typically perceived as white (e.g., sunlight, incandescent light, and fluorescent light). It therefore may be useful to characterize a system based on the amount by which the CIE coordinates of incident light are shifted when the light is transmitted and/or reflected by the system. For example, a system in which light with CIE coordinates of (0.33, 0.33) becomes light with a CIE of (0.30, 0.30) after transmission may be described as causing a color shift of (−0.03, −0.03), or, more generally, (.+−.0.03, .+−.0.03). Thus the color shift caused by a system indicates how “natural” light and viewed items appear to an observer. Embodiments of the invention comprise systems causing color shifts of less than (.+−0.0.05, .+−.0.05) to (.+−.0.02, .+−.0.02). A color balancing component may be used to further reduce color shift, but it is preferred to achieve low color shift using only selective filtering without a color balancing component.

An “ophthalmic system” as used herein includes prescription or non-prescription ophthalmic lenses, e.g., for clear or tinted glasses (or spectacles), sunglasses, contact lenses with and without visibility and/or cosmetic tinting, intra-ocular lenses (IOLs), corneal grafts, corneal tissue, corneal inlays, corneal on-lays, retinal tissue, and electro-active ophthalmic devices and may be treated or processed or combined with other components to provide desired functionalities described in further detail herein. Embodiments of the invention can be formulated so as to allow being applied directly into corneal tissue.

As used herein, an “ophthalmic material” is one commonly used to fabricate an ophthalmic system, such as a corrective lens. Exemplary ophthalmic materials include glass, plastics such as CR-39, Trivex, and polycarbonate materials, as well as MR-7, MR-8, and MR-10, though other materials may be used and are known for various ophthalmic systems.

An ophthalmic system may include a blue blocking component posterior to a color-balancing component. Either of the blue blocking component or the color balancing component may be, or form part of, an ophthalmic component such as a lens. The posterior blue blocking component and anterior color balancing component may be distinct layers on or adjacent to or near a surface or surfaces of an ophthalmic lens. The color-balancing component may reduce or neutralize a yellow or amber tint of the posterior blue blocking component, to produce a cosmetically acceptable appearance. For example, to an external viewer, the ophthalmic system may look clear or mostly clear. For a system user, color perception may be normal or acceptable. Further, because the blue blocking and color balancing tints are not intermixed, wavelengths in the blue light spectrum may be blocked or reduced in intensity and the transmitted intensity of incident light in the ophthalmic system may be at least 80% for unblocked wavelengths.

In order to further protect the human eye from exposure to both harmful high energy visible light wavelengths and UV light and optionally IR light non-ophthalmic applications for embodiments of the invention are also included.

A “non-ophthalmic system” includes any light transmissive structure, excluding ophthalmic lenses, through which light passes on its way to a viewer, as well as skin creams and lotions. By way of example only, non-ophthalmic systems may include: artificial lighting (non-sunlight), diffusers, any type of light bulb, windows, windshields, aircraft windows, instruments, operating devices and other equipment used by ophthalmologists and other eye care professionals to examine the eyes of patients, medical devices, telescopes, binoculars, hunting scopes for rifles, shotguns, and pistols, computer monitors, television sets, camera flashes, virtually any and all electronic devices that emit or transmit visible light, or any type of product or device whereby visible light is emitted or travels through said product or device whereby light from that product or device enters the human eye whether the light is filtered or not by the product or device can be enabled with embodiments of the invention. A non-ophthalmic system may further include dermatological products such as any skin or hair product, suntan and sunscreen products, lip stick, lip balm, anti-ageing products, oils, or acne products. Furthermore, military and space applications also apply as acute and/or chronic exposure to high energy visible light, UV, and also IR can potentially have a deleterious effect on soldiers and astronauts.

Embodiments of the inventions could include by way of example only: any type of windows, or sheet of glass, or any transparent material, automotive windshields, aircraft windows, camera flash bulbs and lenses, any type of artificial lighting fixture (either the fixture or the filament or both), fluorescent lighting, LED lighting or any type of diffuser, medical instruments, surgical instruments, rifle scopes, binoculars, computer monitors, televisions screens, lighted signs or any other item or system whereby light is emitted or is transmitted or passes through filtered or unfiltered.

Embodiments of the invention may enable non-ophthalmic systems. Any non-ophthalmic system whereby, light transmits through or from the non-ophthalmic system can be enabled by the invention. By way of example only, a non-ophthalmic system could include: automobile windows and windshields, aircraft windows and windshields, any type of window, computer monitors, televisions, medical instruments, diagnostic instruments, lighting products, fluorescent lighting, or any type of lighting product or light diffuser.

Any amount of light that reaches the retina can be filtered by embodiments of the invention and can be included in any type of system: ophthalmic, non-ophthalmic, dermatological, or industrial.

Embodiments of the invention include a wide variation in how the selective filter can be added to any system in varying concentrations and/or zones and/or rings and/or layers. For example, in an eyeglass lens the select filter does not necessarily need to be uniform throughout the entire system or in any fixed concentration. An ophthalmic lens could have one or more zones and/or rings and/or layers of varying filter concentration or any combination thereof. In other embodiments the filter can be uniform or mostly uniform throughout the system.

One concern for dyes which selectively filter light in the blue region of the visible light spectrum is that this absorption may affect the color of the light in transmission. Anytime that some wavelengths are filtered relative to others, there will be a difference in the spectrum of light which enters the eye after passing through the lens (filter). Depending on the magnitude of the changes at specific wavelengths, this filtering may cause imperceptible or perceptible changes in color. While each individual's eyes are unique, the effects to an average observer can be estimated by using mathematical models which account the color perception for typical human observers.

There are many dyes, especially within the porphyrin class that one could possibly use as the selective wavelength filter in the invention, however many dyes are not stable or bleach out during the fabrication process. Further, it is imperative that the coating pass CHOCA and/or QUV testing.

Below is the description for CHOCA and QUV tests:

CHOCA (Cycle Humidity Oven/Crosshatch): 3 test cycles (24 hours total), each cycle is 8 hours of exposure to potassium sulfate solution in oven @ 65 C, then 16 hours at ambient conditions. (3 day CHOCA test correspond to 2 years of actual wearing of eyeglasses)

QUV/Accelerated weathering test: 3 test cycles (24 hours total), each cycle is 8 hours of UV exposure @ 60 C, then 4 hours of condensation for 4 hours @ 50 C.

(Lenses are exposed with UV light sources that resemble the solar irradiation, but there is no direct correlation between the QUV and actual environmental conditions)

In one embodiment, there is provided a fabrication process that combines the synergistic balance of Yellowness index, light transmission of the system, selective filtering of light to protect the retina and/or improve contrast, dye formation, dye stability, thickness of the coating, compatibility with substrates to which it is applied, solubility into the resin, refractive index of the dye, protection from UV light; and protection from normal wear and tear.

The selective filter is located within the primer that is applied to the back surface of the lens (ocular surface-closest to the eye) with a scratch resistant coating applied to the front surface of the lens (contra-ocular-furthest from the eye) with a UV inhibiter applied in front or optionally on both the front and rear surface of the lens. The UV inhibitor functions to protect the dye from UV degradation along with reducing UV dose to the eye.

Fabricating the selective high energy visible light coating utilizing tetrakis(2,6-dichloropheyl)porphyrin, meso-Tetra(o-dichlorophenyl)porphine, MTP as the dye are outlined as follows:

In the fabrication of the coating, the UV coating may be on the front surface of the lens, within the polymer and/or selective filter, or on the back surface of the lens, or any possible combination thereof. However in one embodiment the UV blocking is in the front of the lens-furthest from the eye. This allows for protection of the primer and/or dye and also the eye. In another embodiment, applying UV blocking on the rear of the lens-closest to the eye, allows for further reduction of UV light entering the eye by reflection of light from the back surface of the lens.

In other embodiments the dye is dried on the lens surface during the fabrication process by air drying and/or oven drying. UV light should be avoided during this step.

In other embodiments, the dye may require filtering before being applied to the lens.

In other embodiments, during a dip coating process, the front and back surface of a lens is coated with the primer and the dye. In this case the dye on the front surface will fade over time due to UV light exposure to the front primer coating which is unprotected from UV light. This fading will allow for approximately 20% of the dye to fade over a two year period. Therefore, the back surface requires +20% more blockage than the front primer. This embodiment initially artificially elevates the Yellowness Index, which increases eye protection, but as fading occurs over time, the Yellowness Index will decrease.

Embodiments of the invention provide for the YI being variable depending on the intended application. By way of example only, an ophthalmic application such as an eyeglass lens may provide optimal retinal protection and cosmesis with a YI of 5.0 whereby, a non-ophthalmic application such as a window of a home or commercial building may have a much higher YI of 15.0 so as to reduce overall light transmission with an even higher retinal protection level wherein cosmesis is less important than an ophthalmic eyeglass lens.

Embodiments of the invention include one or more dyes designed to filter high energy blue light wavelengths. These dyes may include porphyrins or derivatives with or without Soret bands. The dyes may include one or more peaks based on the intended target wavelengths. The dyes may also vary in slope. Further rings, layers, or zones of filtering can be incorporated into embodiments of the invention. By way of example only, in the non-ophthalmic use of an automotive windshield it may be prudent to incorporate a layer of filtering in the upper horizontal aspect of the front windshield to both reduce glare from the sun and provide higher retinal protection than other parts of the windshield.

Embodiments of the present include the dye tetrakis(2,6-dichlorophenyl)porphyrin or otherwise known as meso-Tetra(o-dichlorophenyl)porphine. The chemical formula is C-44 H-22 CL-8 N-4 with a CAS Number of 37083-37-7. The dye is also known as MTP.

Embodiments of the present invention include UV and/or IR blocking. Embodiments of the invention can be applied to a static focus lens comprising a non changeable color, a static focus lens comprising a changeable color such as, by way of example only, photochromic lens such as Transitions, a dynamic focusing lens comprising a non changeable color, a dynamic focusing lens comprising a changeable color such as, by way of example only, photochromic lens such as Transitions.

Before describing the various embodiments in greater detail, further explanation and definitions shall be provided regarding certain terms that are used throughout the descriptions below and generally used in the art(s) corresponding to the scope of the present disclosure.

Comprising: The term “comprising” herein corresponds to an open-ended limitation. For example, a device comprising features A, B, and C is a device that may, in addition to features A, B, and C have features D, E, F, etc.

Consisting: The term “consisting” herein corresponds to a closed limitation. For example, a group consisting of A, B, C, and D is understood herein to mean that the group is made of elements A, B, C, and D only.

Consisting essentially of: herein, an embodiment consisting essentially of one or more features means that the embodiment necessarily includes those feature(s), but that it is open to having unlisted additional features, provided that these unlisted additional features do not materially affect the basic and novel characteristic(s) of the claimed invention. The term “consisting essentially of” herein is a middle grown between the open limitation format of the “comprising” language and the closed format of the “consisting” language, as described above.

Soret band: The Soret band of a dye is a relatively narrow band of the visible electro-magnetic spectrum located in the blue light region of the spectrum in which the dye has intense absorption of blue light. A Soret peak is thus a local maximum in the Soret band.

CR-39, also known as allyl diglycol carbonate (ADC), is a plastic polymer commonly used in the manufacture of eyeglass lenses.” CR-39 is available from PPG Industries.

MR-7, MR-8, and MR-10 are materials available from Mitsui Chemicals Corporation, Tokyo Japan. These are materials well-known for use as substrates for ophthalmic systems.

Average size: the term “average size,” when used with respect to aggregates of a dye or aggregates of a dye mixture, herein is the arithmetic average (i.e. the mean) of all the diameters of the aggregates.

One way to ensure that a dye or dye mixture has average aggregate size less than a particular quantity is to pass the solution containing the dye or dye mixture through a filter having a mesh size corresponding to the desired average size. Such filtration will generally result in a mixture where the average size is somewhat below the mesh size.

Before turning to the embodiments described below, it should be noted that generally, dyes desirable for achieving selective blue blocking such as porphyrin and porphyrin derivatives, when loaded in solvents conventionally used in the ophthalmic industry, may have aggregate sizes larger than 10 micrometers. As such, when filtration is performed with a filter having mesh size less than 10 micrometers, the filtration has low or negligible yield. If, alternatively, filtration is bypassed altogether, large aggregate sizes cause large haze values, which also hinder light transmission performance, in addition to making the system which incorporates the dye cosmetically unappealing. Using unconventional solvents as disclosed herein allows the reduction of aggregate size, thus permitting high yield filtration and low haze values. Several example embodiments that have these characteristics are described below.

### Example 1

### CR-39 Lenses

Fabrication steps for making CR-39 lenses with selective light blockage coating on the back of lenses are as follows: #1) utilize a Semi-finished lens blank comprising a hard coat (front surface), #2) surface and polish the backside to optical power needed of lens, #3) add UV protection to the lens, #4) prepare HPO-dye package in the primer coating. This includes measuring the proper amount of a dye or dye mixture depending on what wavelength range is needed to be blocked and the % of required blockage. For instance, for a selective light blockage in the spectral range 430+/−20 nm, a single component dye package comprising MTP dye is sufficient. The steps further include methods to: dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes.

The steps further including adding the prepared solution of dye(s) to the primer coating, ultrasonicating and heating (up-to 50 C) the primer coating with the added dye(s) for 30 min. Filtrate the solution through appropriate filters (e.g. 1 or 1.5 μm Nylon filters). Further, the fabrication steps include: #5) adding primer coating of HPO-dye package to the backside of the lens, #6) dry the coating of #5, #7) adding Hard Scratch Resistant Coating (HC) to the backside of the lens, and #8) curing coating of #7.

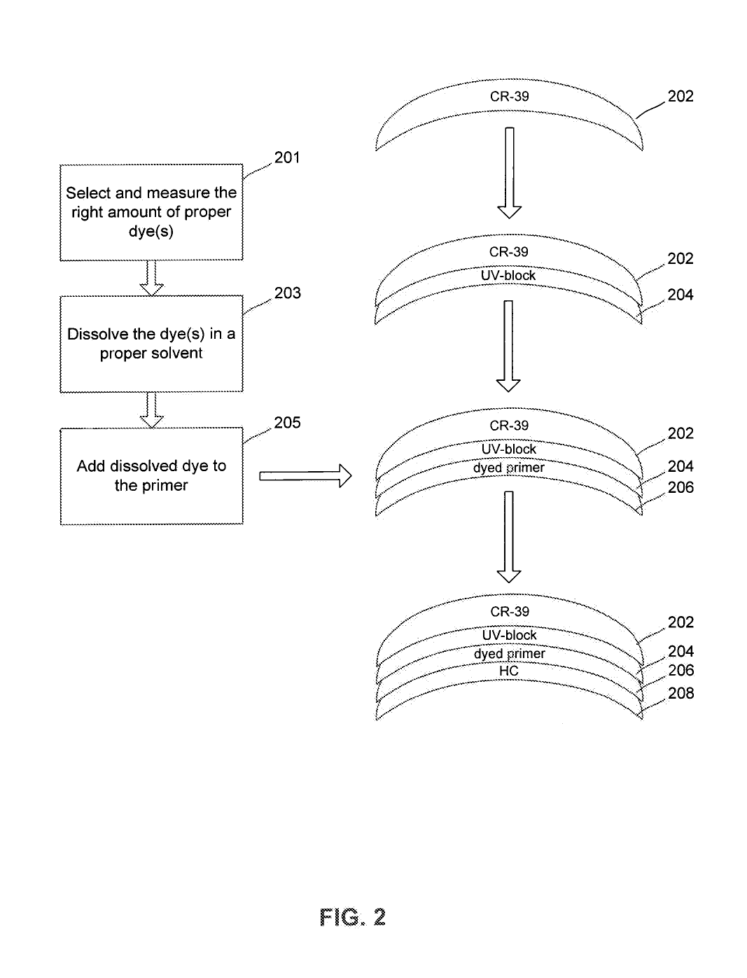

FIG. 2 describes a detailed description of the fabrication method described above, according to an embodiment of the invention.

The method comprises providing a solution that contains a dye or a dye mixture, either of which is referred hereinafter as “the dye” for clarity. Providing the solution comprises selecting the dye and measuring an amount of the dye which is then dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. However, solvents with higher polarity index (e.g. P>3.0) may be used. Further, chlorinated solvents or mixtures thereof may also be used in embodiments where the dye is (MTP). In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication, in general, may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method further comprises providing a substrate 202. In this embodiment, substrate 202 is a semi-finished lens blank, for example CR-39, and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 202 further comprises surfacing (or machining) and polishing at least one side of substrate 202. In case substrate 202 is an ophthalmic substrate, such as for example, a CR-39 semi-finished lens blank, machining and polishing provide a predetermined optical power which is prescribed to the patient.

Substrate 202 is then fitted with a UV-blocking coating 204. UV-blocking coating 204 may be disposed on substrate 202 using spin-coating and curing, or any other methods suitable for applying UV protection to a substrate. For example, UV-blocking coating 204 may be disposed on substrate 202 by dipping substrate 202 in a solution containing a UV-blocking dye.

Subsequently, the dye-loaded primer coating formulation is disposed on the backside of substrate 202, namely on UV-blocking coating 204. Air drying or a short thermal baking may be used to cure the applied dye-loaded primer coating formulation to form selective blue-blocking coating 206. Selective blue-blocking coating 206 comprises the dye and selectively inhibits the transmission of blue light. A hard scratch resistant coating 208 is then disposed and cured on selective blue-blocking coating 206. Disposing and curing hard scratch resistant coating 208 may be achieved using deposition and coating methods similar to those described above.

By way of example only, the dye, when incorporated in substrate 202's optical path as described above, absorbs 5-50% of at least one wavelength of light in the blue light wavelength range of 400 nm to 500 nm. In alternate embodiments, the dye, when incorporated in substrate 202's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

### Example 2

### Polycarbonate (PC) Lenses

Fabrication steps for making polycarbonate (PC) lenses with selective light blockage coating on the back of lenses are given below:

#1) Utilize a Semi-finished lens blank comprising a hard coat (front surface).

#2) Surface and Polish backside to optical power needed of lens.

#3) Prepare HPO-dye package in the primer coating:

Measure the proper amount of a dye or dye mixture depending on what wavelength range is needed to be blocked and the % of required blockage. For instance, for a selective light blockage in the spectral range 430+/−20 nm, a single component dye package comprising MTP dye is sufficient.

Dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes.

Add the prepared solution of dye(s) to the primer coating.

Ultrasonicate and heat (up-to 50 C) the primer coating with the added dye(s) for 30 min. Filtrate the solution through appropriate filters (e.g. 1 or 1.5 μm Nylon filters). #4) Add primer coating of HPO-dye package, prepared in step #3, to backside of lens. #5) Dry coating of #4. #6) Add Hard Scratch Resistant Coating (HC) to backside of lens. #7) Cure coating of #6.

FIG. 3 describes a detailed description of the fabrication method described above, according to an embodiment of the invention.

The method comprises providing a solution that contains a dye or a dye mixture, either of which is referred hereinafter as “the dye” for clarity. Providing the solution comprises selecting the dye and measuring an amount of the dye which is then dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication, in general, may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method further comprises providing a substrate 302. In this embodiment, substrate 202 is a semi-finished lens blank, for example polycarbonate (PC), and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 302 further comprises surfacing (or machining) and polishing at least one side of substrate 302. In case substrate 302 is an ophthalmic substrate, such as for example, a PC semi-finished lens blank, machining and polishing provide a predetermined optical power which is prescribed to the patient. Contrary to the embodiment described in FIG. 2, the present embodiment of the method does not include disposing a UV-blocking coating on substrate 302.

Rather, the dye-loaded primer coating formulation is disposed on the backside of substrate 302 directly. Air drying or a short thermal baking may be used to cure the applied dye-loaded primer coating formulation to form selective blue-blocking coating 306. Selective blue-blocking coating 306 comprises the dye and selectively inhibits the transmission of blue light. A hard scratch resistant coating 308 is then disposed and cured on selective blue-blocking coating 206. Disposing and curing hard scratch resistant coating 308 may be achieved using deposition and coating methods similar to those described above.

By way of example only, the dye, when incorporated in substrate 302's optical path as described above, absorbs 5-50% of at least one wavelength of light in the blue light wavelength range of 400 nm to 500 nm. In alternate embodiments, the dye, when incorporated in substrate 302's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

### Example 3

### MR-8 Lenses

Fabrication process for making MR-8 lenses with selective light blockage coating on the back of lenses can be done in two different ways depending on the required UV protection.

3.1. MR-8 Lenses without additional UV block. Due to the intrinsic UV-blocking character of MR-8 material, MR-8 lenses can partially block the UV-A and UV-B light. Fabrication steps for MR-8 lenses without adding additional UV block are as follows: #1) Utilize a Semi-finished lens blank comprising a hard coat (front surface). #2) Surface and Polish backside to optical power needed of lens. #3) Prepare HPO-dye package in the primer coating:

Measure the proper amount of a dye or dye mixture depending on what wavelength range is needed to be blocked and the % of required blockage. For instance for a selective light blockage in the spectral range 430+/−20 nm, a single component dye package comprising MTP dye is sufficient.

Dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes.

Add the prepared solution of dye(s) to the primer coating.

Ultrasonicate and heat (up-to 50 C) the primer coating with the added dye(s) for 30 min. Filtrate the solution through appropriate filters (e.g. 1 or 1.5 μm Nylon filters). #4) Add primer coating of HPO-dye package to backside of lens. #5) Dry coating of #4. #6) Add Hard Scratch Resistant Coating (HC) to backside of lens. #7) Cure coating of #6.

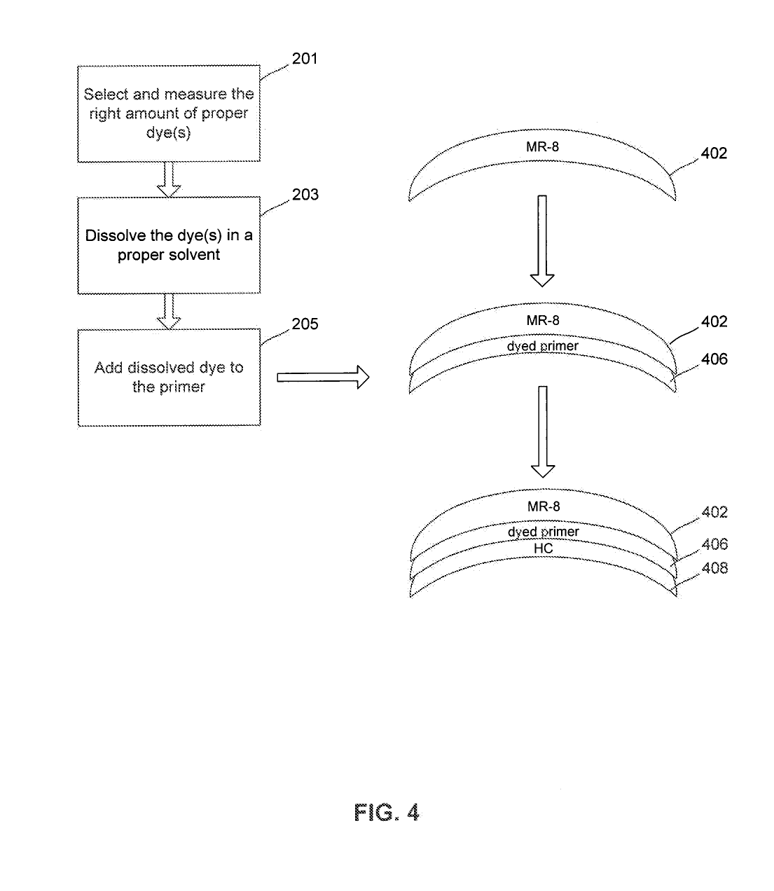

FIG. 4 describes a detailed description of the fabrication method described above, according to an embodiment of the invention.

The method comprises providing a solution that contains a dye or a dye mixture, either of which is referred hereinafter as “the dye” for clarity. Providing the solution comprises selecting the dye and measuring an amount of the dye which is then dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication, in general, may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method farther comprises providing a substrate 402. In this embodiment, substrate 402 is a semi-finished lens blank, for example MR-8, and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 402 further comprises surfacing (or machining) and polishing at least one side of substrate 402. In case substrate 402 is an ophthalmic substrate, such as for example, a MR-8 semi-finished lens blank, machining and polishing provide a predetermined optical power which is prescribed to the patient. Contrary to the embodiment described in FIG. 2, the present embodiment of the method does not include disposing a UV-blocking coating on substrate 402.

Rather, the dye-loaded primer coating formulation is disposed on the backside of substrate 402 directly. Air drying or a short thermal baking may be used to cure the applied dye-loaded primer coating formulation to form selective blue-blocking coating 406. Selective blue-blocking coating 406 comprises the dye and selectively inhibits the transmission of blue light. A hard scratch resistant coating 408 is then disposed and cured on selective blue-blocking coating 406. Disposing and curing hard scratch resistant coating 408 may be achieved using deposition and coating methods similar to those described above.

By way of example only, the dye, when incorporated in substrate 402's optical path as described above, absorbs 5-50% of at least one wavelength of light in the blue light wavelength range of 400 nm to 500 nm. In alternate embodiments, the dye, when incorporated in substrate 402's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

3.2. MR-8 Lenses with additional UV block Fabrication steps for MR-8 lenses with additional UV block are given below: #1) Utilize a Semi-finished lens blank comprising a hard coat (front surface). #2) Surface and Polish backside to optical power needed of lens. #3) Add UV protection to the lens. It can be added in two ways: By dipping in warm UV-dye blocking bath, or By spin-coating of solution containing UV-blocking dye. #4) Prepare HPO-dye package in the primer coating: Measure the proper amount of a dye or dye mixture depending on what wavelength range is needed to be blocked and the % of required blockage. For instance for a selective light blockage in the spectral range 430+/−20 nm, a single component dye package comprising MTP dye is sufficient.

Dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes. Add the prepared solution of dye(s) to the primer coating. Ultrasonicate and heat (up-to 50 C) the primer coating with the added dye(s) for 30 min. Filtrate the solution through appropriate filters (e.g. 1 or 1.5 μm Nylon filters). #5) Add primer coating of HPO-dye package to backside of lens. #6) Dry coating of #5. #7) Add Hard Scratch Resistant Coating (HC) to backside of lens. #8) Cure coating of #7.

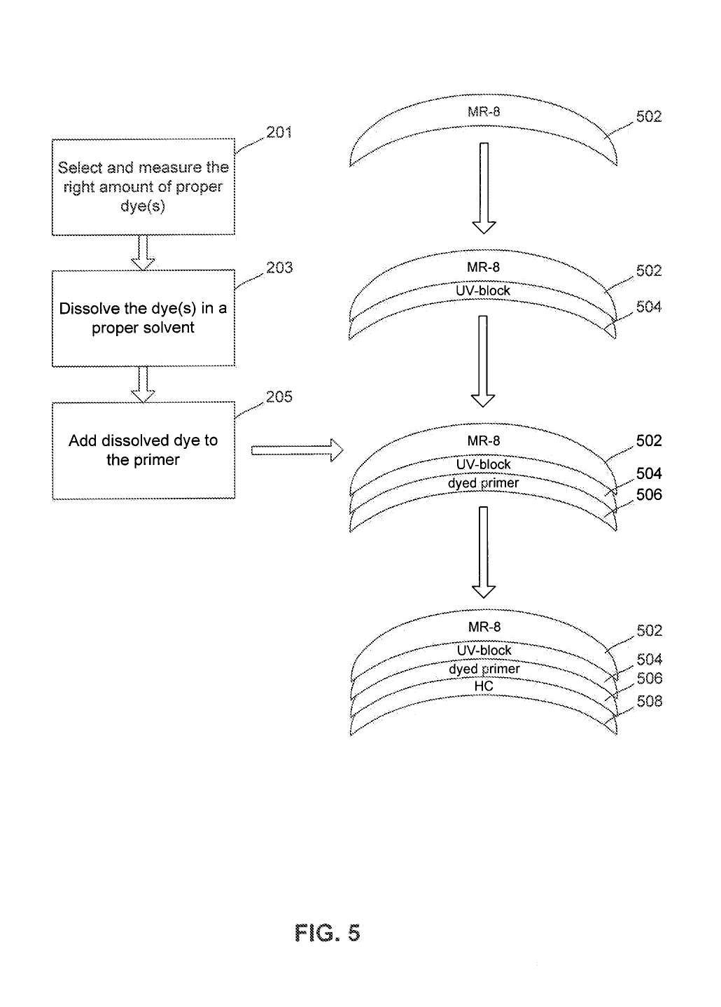

FIG. 5 illustrates a fabrication method according to an embodiment of the invention. The method comprises providing a solution that contains a dye or a dye mixture, either of which is referred hereinafter as “the dye” for clarity. Providing the solution comprises selecting the dye and measuring an amount of the dye which is then dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method further comprises providing a substrate 502. In this embodiment, substrate 502 is a semi-finished lens blank, for example MR-8, and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 502 further comprises surfacing (or machining) and polishing at least one side of substrate 502. In case substrate 502 is an ophthalmic substrate, such as for example, a MR-8 semi-finished lens blank, machining and polishing provide a predetermined optical power which is prescribed to the patient.

Substrate 502 is then fitted with a UV-blocking coating 504. UV-blocking coating 504 may be disposed on substrate 502 using spin-coating and curing, or any other methods suitable for applying UV protection to a substrate. For example, UV-blocking coating 204 may be disposed on substrate 502 by dipping substrate 502 in a solution containing a UV-blocking dye.

Subsequently, the dye-loaded primer coating formulation is disposed on the backside of substrate 502, namely on UV-blocking coating 504. Air drying or a short thermal baking may be used to cure the applied dye-loaded primer coating formulation to form selective blue-blocking coating 506. Selective blue-blocking coating 506 comprises the dye and selectively inhibits the transmission of blue light. A hard scratch resistant coating 208 is then disposed and cured on selective blue-blocking coating 506. Disposing and curing hard scratch resistant coating 508 may be achieved using deposition and coating method similar to those described above.

By way of example only, the dye, when incorporated in substrate 502's optical path as described above, absorbs 5-50% of at least one wavelength of light in the blue light wavelength range of 400 nm to 500 nm. In alternate embodiments, the dye, when incorporated in substrate 502's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

### Example 4

### MR-7 Lenses

Fabrication steps for making MR-7 lenses with selective light blockage coating on the back of lenses are given below: #1) Utilize a Semi-finished lens blank comprising a hard coat (front surface). #2) Surface and Polish backside to optical power needed of lens. #3) Prepare HPO-dye package in the primer coating: Measure the proper amount of a dye or dye mixture depending on what wavelength range is needed to be blocked and the % of required blockage. For instance, for a selective light blockage in the spectral range 430+/−20 nm, a single component dye package comprising MTP dye is sufficient.

Dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes.

Add the prepared solution of dye(s) to the primer coating. Ultrasonicate and heat (up-to 50 C) the primer coating with the added dye(s) for 30 min. Filtrate the solution through appropriate filters (e.g. 1 or 1.5 μm Nylon filters). #4) Add primer coating of HPO-dye package, prepared in step #3, to backside of lens. #5) Dry coating of #4. #6) Add Hard Scratch Resistant Coating (HC) to backside of lens. #7) Cure coating of #6.

FIG. 6 describes a detailed description of the fabrication method described above, according to an embodiment of the invention.

The method comprises providing a solution that contains a dye or a dye mixture, either of which is referred hereinafter as “the dye” for clarity. Providing the solution comprises selecting the dye and measuring an amount of the dye which is then dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication, in general, may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method further comprises providing a substrate 602. In this embodiment, substrate 602 is a semi-finished lens blank, for example MR-7, and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 602 further comprises surfacing (or machining) and polishing at least one side of substrate 602. In case substrate 602 is an ophthalmic substrate, such as for example, a MR-7 semi-finished lens blank, machining and polishing provide a predetermined optical power which is prescribed to the patient. Contrary to the embodiment described in FIG. 2, the present embodiment of the method does not include disposing a UV-blocking coating on substrate 602.

Rather, the dye-loaded primer coating formulation is disposed on the backside of substrate 602 directly. Air drying or a short thermal baking may be used to cure the applied dye-loaded primer coating formulation to form selective blue-blocking coating 606. Selective blue-blocking coating 606 comprises the dye and selectively inhibits the transmission of blue light. A hard scratch resistant coating 608 is then disposed and cured on selective blue-blocking coating 606. Disposing and curing hard scratch resistant coating 608 may be achieved using deposition and coating methods similar to those described above.

By way of example only, the dye, when incorporated in substrate 602's optical path as described above, absorbs 5-50% of at least one wavelength of light in the blue light wavelength range of 400 nm to 500 nm. In alternate embodiments, the dye, when incorporated in substrate 602's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

### Example 5

### MR-10 Lenses

Fabrication steps for making MR-10 lenses with selective light blockage coating on the back of lenses are given below: #1) Utilize a Semi-finished lens blank comprising a hard coat (front surface). #2) Surface and Polish backside to optical power needed of lens. #3) Prepare HPO-dye package in the primer coating: Measure the proper amount of a dye or dye mixture depending on what wavelength range is needed to be blocked and the % of required blockage. For instance, for a selective light blockage in the spectral range 430□20 nm, a single component dye package comprising MTP dye is sufficient.

Dissolve the dye/dye package in appropriate solvent or solvent mixture. For instance, for MTP dye, cyclopentanone, cyclohexanone, methyl ethyl ketone, DMSO, DMF, and many other solvents or their combination work well. Any handbook on organic solvents (e.g. please, see the organic solvents' table at http://murov.info/orgsolvents.htm) has the data on organic solvents and their properties. In general, solvents with moderate and high polarity work well for MTP and other similar dyes. Add the prepared solution of dye(s) to the primer coating.

Ultrasonicate and heat (up-to 50 C) the primer coating with the added dye(s) for 30 min. Filtrate the solution through appropriate filters (e.g. 1 or 1.5 μm Nylon filters). #4) Add primer coating of HPO-dye package, prepared in step #3, to backside of lens. #5) Dry coating of #4. #6) Add Hard Scratch Resistant Coating (HC) to backside of lens. #7) Cure coating of #6.

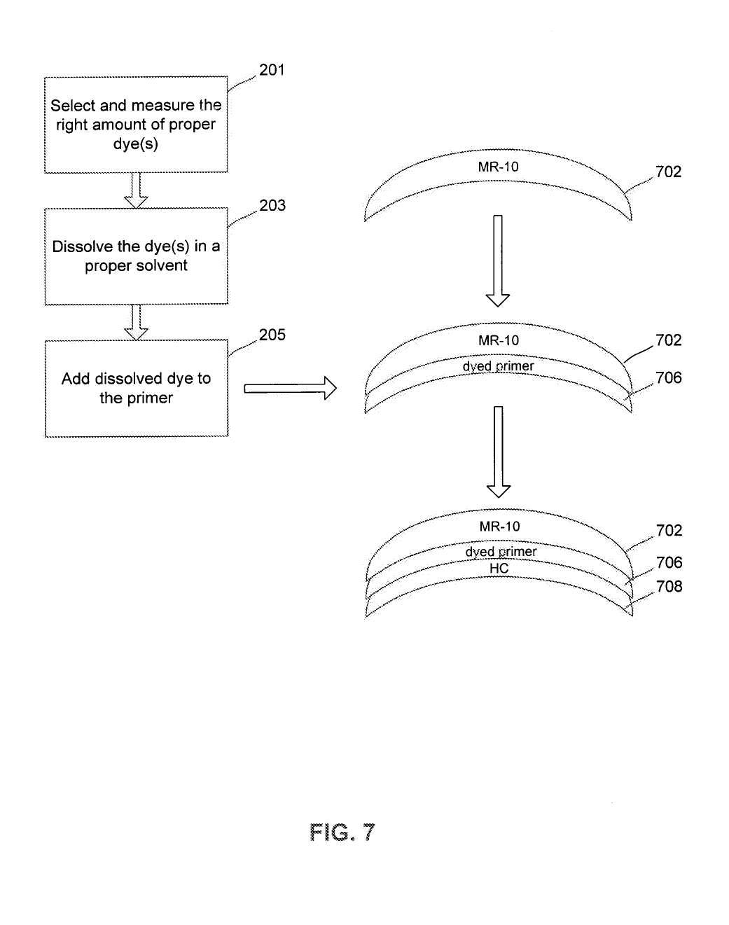

FIG. 7 describes a detailed description of the fabrication method described above, according to an embodiment of the invention.

The method comprises providing a solution that contains a dye or a dye mixture, either of which is referred hereinafter as “the dye” for clarity. Providing the solution comprises selecting the dye and measuring an amount of the dye which is then, dissolved in a solvent (step 201). In this example embodiment the dye may be MTP (or it may comprise MTP), and by way of example only, 1 g of the dye may be dissolved in 100 g of solvent, thus providing a concentration of 1 wt %. In this example, the solvent may be chloroform. In alternate embodiments, the solvent may consist essentially of chloroform. To better promote homogeneity, dissolving the dye in the solvent (step 203) may include ultrasonication.

Following the dissolution of the dye in the solvent (step 203), the dye-laden solution is loaded in a primer coating formulation (step 205). The loaded primer coating formulation is then ultrasonicated and filtrated. Ultrasonication, in general, may be carried out in a temperature controlled environment, for example in an environment wherein the temperature may be set to 50 degrees C. or less. The loaded primer coating formulation is then filtrated using, by way of example only, using a 5-micrometer filter, or preferably, a 1 or 1.5-micrometer filter. The filter in either case may be a Nylon filter. Prior to filtration, the loaded primer coating formulation or the solution may comprise aggregates of the dye that are greater than 10 micrometer in average size. After filtration, the aggregates' average size may be less than 5 micrometers, preferably less than 1.5 micrometers, and more preferably less than 1 micrometer.

In addition to steps 201, 203, and 205 described above, the method further comprises providing a substrate 702. In this embodiment, substrate 702 is a semi-finished lens blank, for example MR-10, and the method is directed towards fabricating an ophthalmic system. In alternate embodiments, however, the method may comprise providing a non-ophthalmic substrate, such as (by example only) one of a window glass, a computer screen, and a skin cream or lotion. One of skill in the art will readily understand that the method according to this embodiment may apply to either ophthalmic or non-ophthalmic substrates.

Providing substrate 702 further comprises surfacing (or machining) and polishing at least one side of substrate 702. In case substrate 702 is an ophthalmic substrate, such as for example, a MR-10 semi-finished lens blank, machining and polishing provide a predetermined optical power which is prescribed to the patient. Contrary to the embodiment described in FIG. 2, the present embodiment of the method does not include disposing a UV-blocking coating on substrate 702.

Rather, the dye-loaded primer coating formulation is disposed on the backside of substrate 702 directly. Air drying or a short thermal baking may be used to cure the applied dye-loaded primer coating formulation to form selective blue-blocking coating 706. Selective blue-blocking coating 706 comprises the dye and selectively inhibits the transmission of blue light. A hard scratch resistant coating 708 is then disposed and cured on selective blue-blocking coating 706. Disposing and curing hard scratch resistant coating 708 may be achieved using deposition and coating methods similar to those described above.

By way of example only, the dye, when incorporated in substrate 702's optical path as described above, absorbs 5-50% of at least one wavelength of light in the blue light wavelength range of 400 nm to 500 nm. In alternate embodiments, the dye, when incorporated in substrate 702's optical path absorbs 20-50% of at least one wavelength of light in the blue wavelength rage of 400 nm to 500 nm. Moreover, the absorption spectrum of the dye within the range 400 nm to 500 may have at least one absorption peak. For example, the peak may be located at the at least one wavelength mentioned above. In some embodiments, the absorption peak may be a Soret peak, and it may have a full-width at half-maximum less than 60 nm. In some embodiments, it may have a full-width at half-maximum less than 40 nm.

Furthermore, the method provides an ophthalmic system which has a yellowness index of less than 15. In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less. In alternate embodiments, the method provides an ophthalmic system in which visible light transmission through the ophthalmic system is 80% or greater, preferably 85% or greater, or more preferably 90% or greater.

Embodiments disclosed herein show selective blue blocking accomplished via a dye. Such selective blue blocking may be supplemented by other blue blocking mechanisms, such as an reflective coating, a multi-layer dielectric stack, or other blue blocking mechanisms. Any combinations of a dye or dye mixture, a multi-layer dielectric stack, a reflective coating, or other blue blocking may be used to achieve selective blue blocking. These combinations may take advantage of the strengths of each individual blue blocking mechanism, while diffusing weaknesses. For example, spectacle lenses that incorporate blue blocking via a reflective mechanism may appear bluish to those other than the wearer. Reducing such reflection and supplementing with blue blocking via a dye can reduce this bluish appearance.

Individual contributions of UV-block, HPO dyed primer and hardcoat to the Yellow Index (YI) are given in Table 2, while the total YI values for different surfaced lenses coated according to the above fabrication steps are given in Tables 3-9.

TABLE 2
Individual contributions of UV-block, HPO dyed primer
and hardcoat to the Yellow Index (YI).

| Coating | individual YI contribution |
|---|---|
| UV block (1 side) | 1.0-2.0 |
| HPO dyed primer | 1.0-12.0 |
| Clear hardcoat (both sides) | 1.0-2.0 |

TABLE 3
Total Yellow Index (YI) values for CR-39 lenses coated with
UV block, HPO dyed primer and low index hardcoat.

| CR-39 lenses | total YI |
|---|---|
| CR-39 surfaced lens | 0.5 |
| CR39 + CC side UV block | 1.5-2.0 |
| CR39 + CC side UV block + CC side HPO dyed primer | 3.0-13.0 |
| CR39 + CC side UV + CC side HPO dyed primer + clear | 4.0-15.0 |
| hardcoat | |

TABLE 4
Total Yellow Index (YI) values for PC lenses coated
with HPO dyed primer and low index hardcoat.

| Polycarbonate (PC) lenses | total YI |
|---|---|
| PC surfaced lens | 1.1 |
| PC + CC side HPO dyed primer | 2.0-13.0 |
| PC + CC side HPO dyed primer + clear hardcoat | 3.0-15.0 |

TABLE 5
Total Yellow Index (YI) values for MR-8 lenses coated
with HPO dyed primer and low index hardcoat.

| MR-8 lenses | total YI |
|---|---|
| MR-8 surfaced lens | 0.5 |
| MR-8 + CC side HPO dyed primer | 1.5-13.0 |
| MR-8 + CC side HPO dyed primer + clear hardcoat | 2.5-15.0 |

TABLE 6
Total Yellow Index (YI) values for MR-8 lenses coated with
UV block, HPO dyed primer and low index hardcoat.

| MR-8 lenses | total YI |
|---|---|
| MR-8 surfaced lens | 0.5 |
| MR-8 + CC side UV block | 1.5-2.0 |
| MR-8 + CC side HPO dyed primer | 3.0-13.0 |
| MR-8 + CC side HPO dyed primer + clear hardcoat | 4.0-15.0 |

TABLE 7
Total Yellow Index (YI) values for MR-8 lenses coated
with UV block, HPO dyed primer and low index hardcoat.

| MR-7 lenses | total YI |
|---|---|
| MR-7 surfaced lens | 0.8 |
| MR-7 + CC side HPO dyed primer | 1.5-13.0 |
| MR-7 + CC side HPO dyed primer + clear hardcoat | 2.5-15.0 |

TABLE 8
Total Yellow Index (YI) values for MR-8 lenses coated
with UV block, HPO dyed primer and low index hardcoat.

| MR-10 | total YI |
|---|---|
| MR-10 surfaced lens | 1.8 |
| MR-10 + CC side HPO dyed primer | 3.0-13.0 |
| MR-10 + CC side HPO dyed primer + clear hardcoat | 4.0-15.0 |

TABLE 9
Selective 430 ± 20 nm light blockage vs. total Yellow Index
for lenses of different lens materials coated with HPO dyed primer
followed by appropriate clear hard coat.

| | total YI |
|---|---|
| CR-39 lenses: selective 430 ± 20 nm light blockage (%) | |
| 13.8 | 6.51 |
| 15.6 | 7.52 |
| 22.6 | 10.9 |
| Polycarbonate (PC) lenses: selective 430 ± 20 nm light blockage | |
| (%) | |
| 8.2 | 2.34 |
| 9.8 | 3.38 |
| 10.2 | 4.21 |
| MR-8 lenses: selective 430 ± 20 nm light blockage (%) | |
| 13.1 | 6.34 |
| 15.6 | 7.45 |
| 17.8 | 8.51 |
| MR-7 lenses: selective 430 ± 20 nm light blockage (%) | |
| 11.8 | 5.89 |
| 14.4 | 6.81 |
| 16.8 | 8.51 |
| 18.8 | 9.61 |
| MR-10 lenses: selective 430 ± 20 nm light blockage (%) | |
| 18.1 | 6.56 |
| 19.4 | 7.92 |
| 23.4 | 9.95 |
| 24.2 | 10.32 |

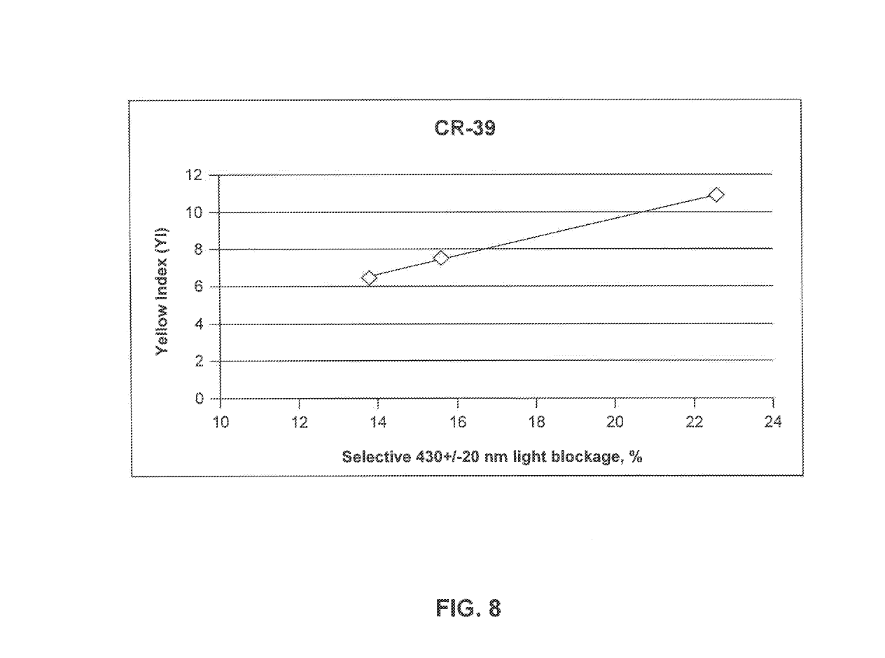

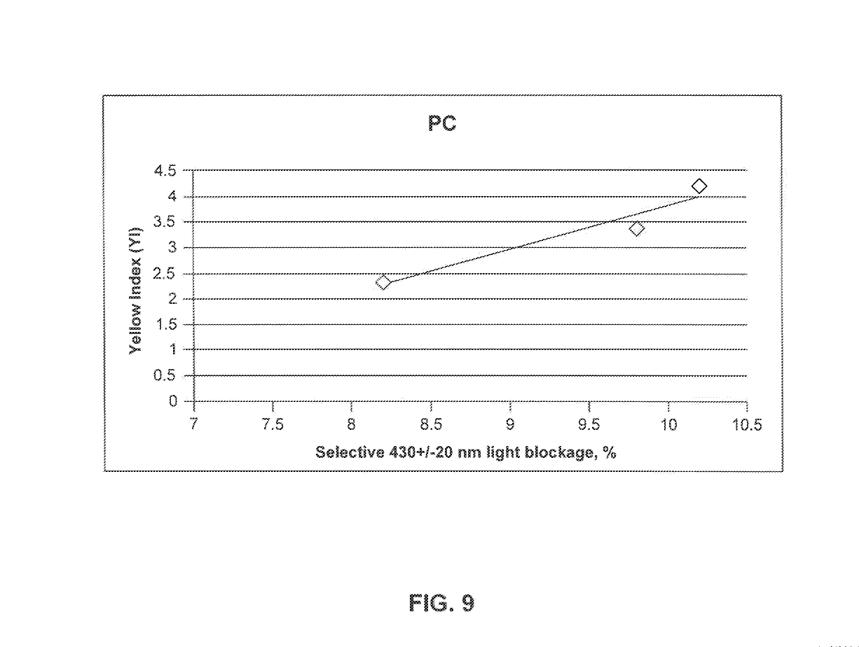

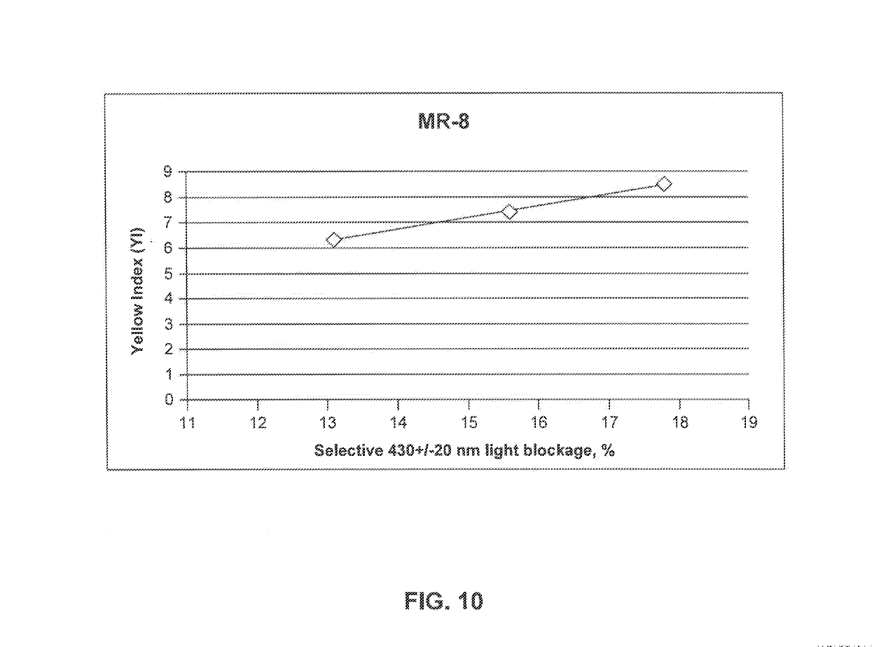

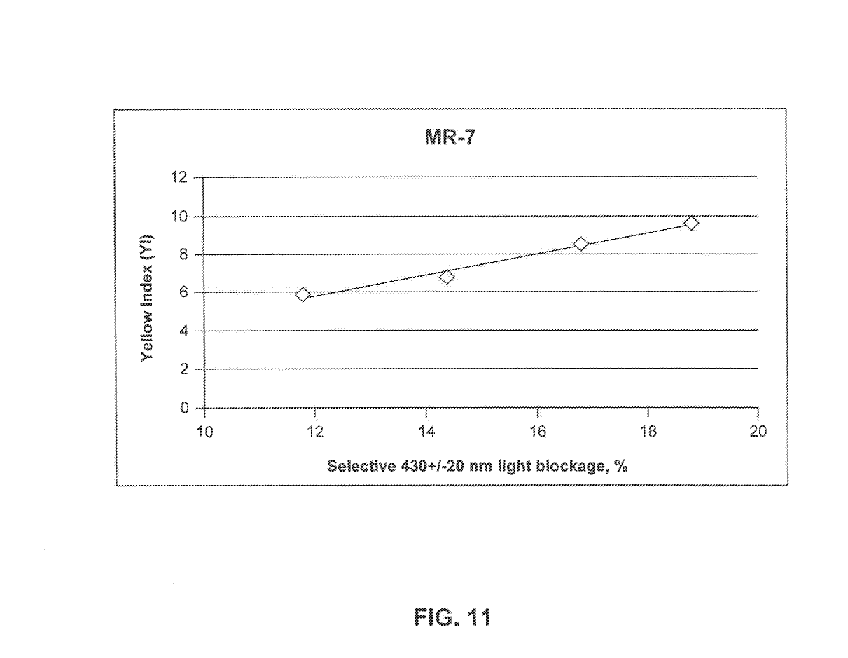

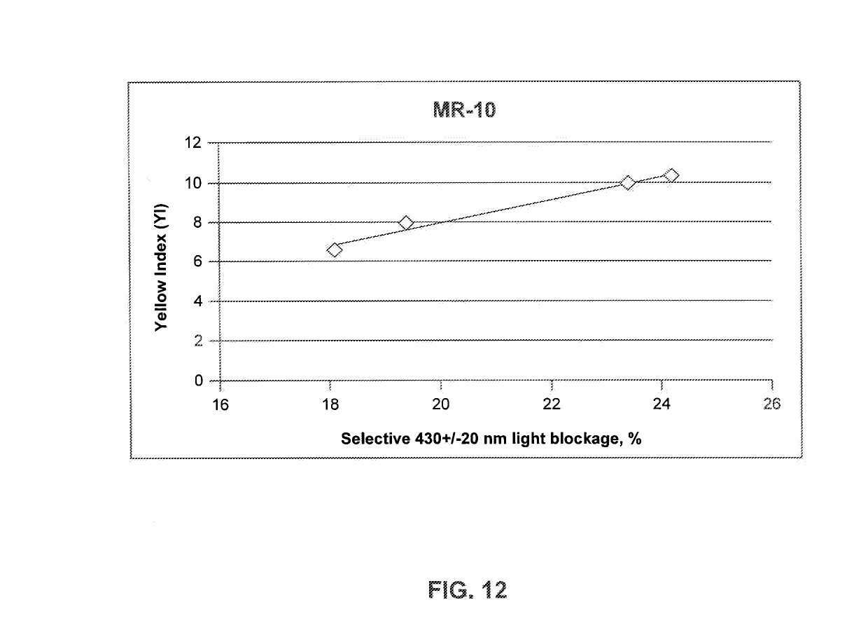

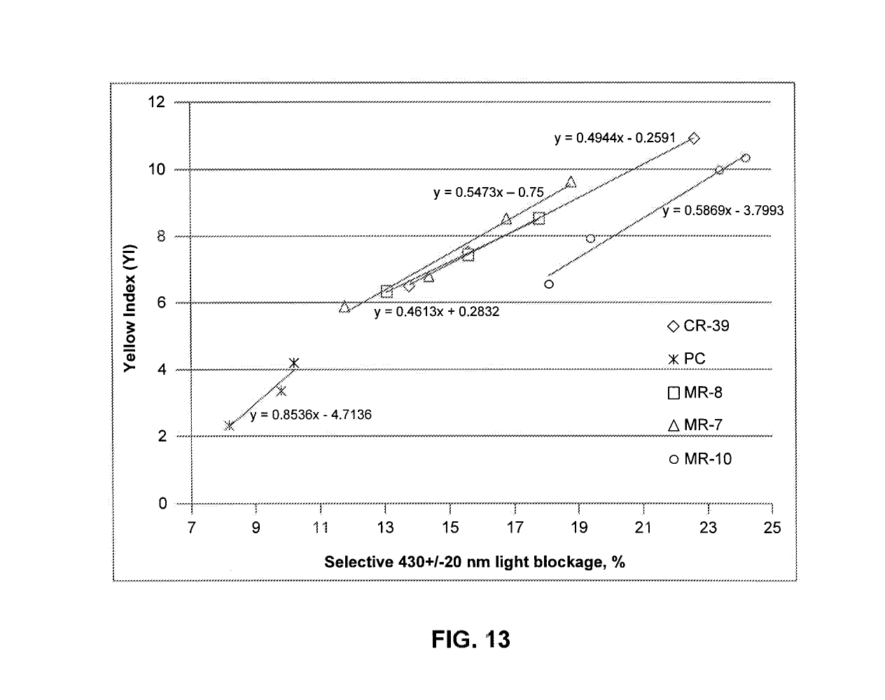

FIGS. 8-13 illustrate selective 430±20 nm light blockage vs. total Yellow Index for lenses coated with HPO dyed primer followed by appropriate clear hard coat.

### Example 6

### CR39 Protected by Front and Back UV Inhibitor

This embodiment of the invention provides a process and resultant high energy selective blue light filtered CR39 lens whereby the UV inhibitor coating is applied such to protect the selective blue light filter dye from UV light.

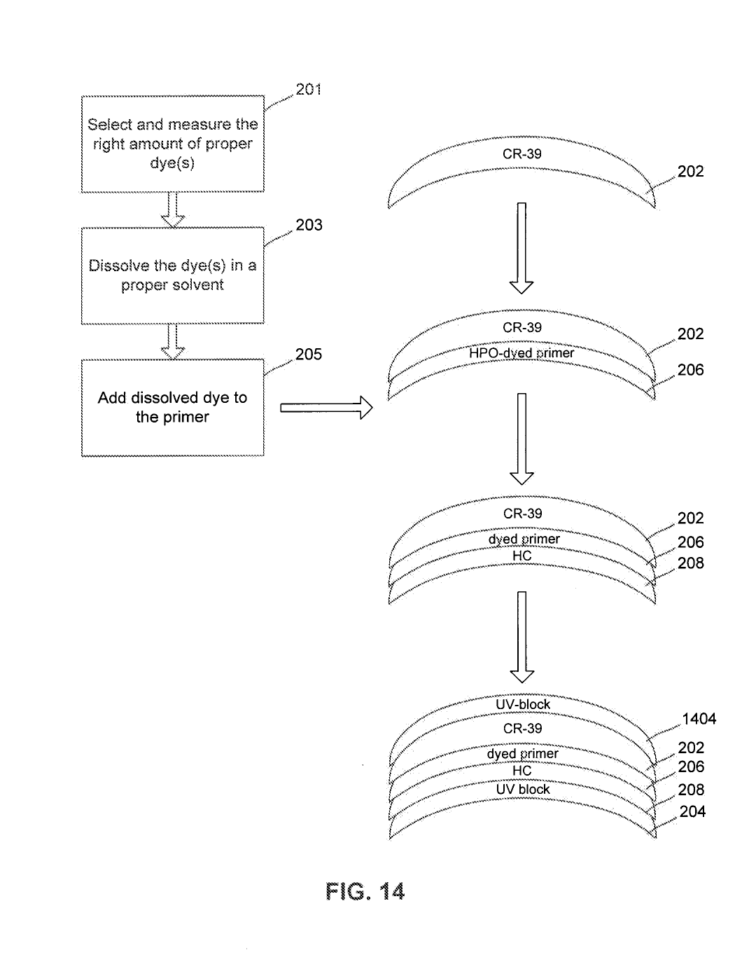

FIG. 14 illustrates a fabrication method according to an embodiment of the invention and as described in Example 6.

The method of FIG. 14 is similar to the method of FIG. 2, and for clarity, common steps are not repeated. The method of FIG. 14 differs from the method of FIG. 2 in two ways. Firstly, in the method of FIG. 14, UV-blocking coating 204 is disposed on the hard scratch resistant coating 208. Secondly, in the method FIG. 14, an additional U-V blocking coating 1404 is provided on the other side of substrate 202.

### Example 7

### MR-8 Protected UV Inhibitor

This embodiment of the invention provides a process and resultant high energy selective blue light filtered MR 8 lens whereby the UV inhibitor coating is applied such to protect the selective blue light filter dye from UV light.

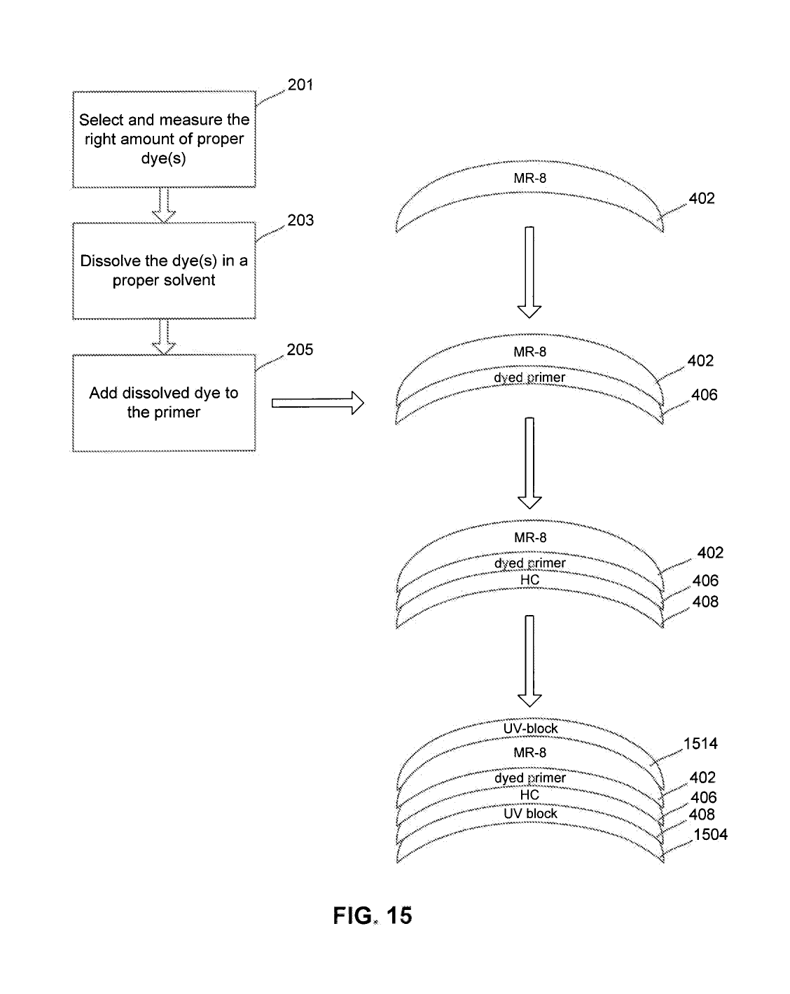

FIG. 15 illustrates a fabrication method according to an embodiment of the invention and as described in Example 7.

The method of FIG. 15 is similar to the method of FIG. 4, and for clarity, common steps are not repeated. The method of FIG. 15 differs from the method of FIG. 4 in that additional U-V blocking coatings 1504 and 1514 are provided on either side of the ophthalmic system after depositing hard scratch resistant coating 208.

A most important feature in the fabrication of the coating is to include a UV inhibitor in front of the dye-closer to the contra-ocular surface than the dye itself so as to protect the potential breakdown of the dye over time due to UV light exposure. In other embodiments, the UV inhibitor can be mixed with the dye to protect the dye, and in some embodiments the UV inhibitor can be placed closer to the ocular surface of the dye than the dye itself. And in other embodiments the UV inhibitor can be placed on more than one surface or within the dye.

### Example 8

### CR39 or MP8 Lenses Coated with Dyed Primer by Dip Coating Method on Both Surfaces (Front and Back) Followed by UV Blocker on Both Surfaces (Front and Back)

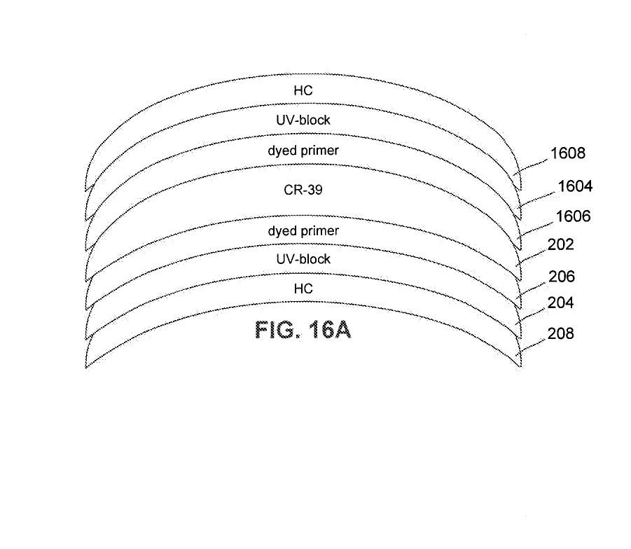

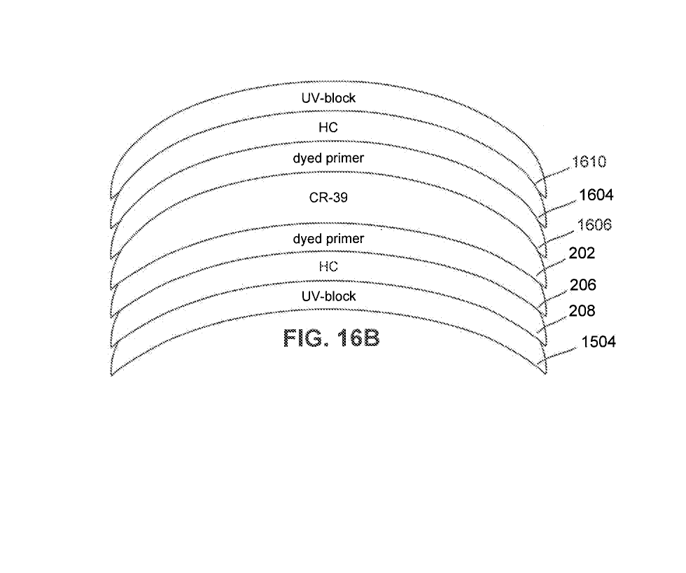

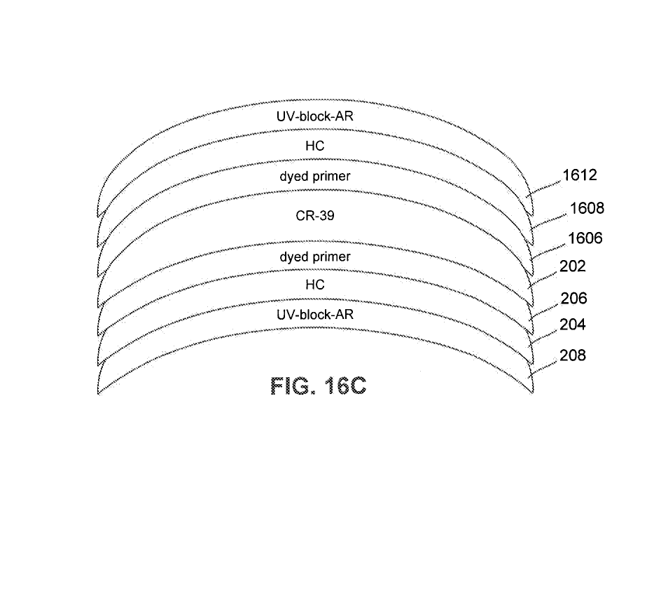

FIGS. 16A, 16B, and 16C each illustrates an ophthalmic system according to embodiments of the invention and as described in Example 8.

In either embodiment, U-V blocking may be provided on either side of the substrate 202 (e.g. UV-blocking layers 1604 and 204) but in arrangement that protects selective blue-blocking layer 206. Moreover, as previously discussed, an additional selective blue-blocking layer 1606 may be added (1606). In addition, an extra hard scratch resistant coating 1608 may be provided. It should also be noted that U-V blocking layers may also be anti-reflective (1612).

### Example 9

### All Lenses that are Made of Inherently UV-Blocking Material (PC, MR7, MR10) Coated with Dyed Primer by Dip Coating Method on Both Surfaces (Front and Back)

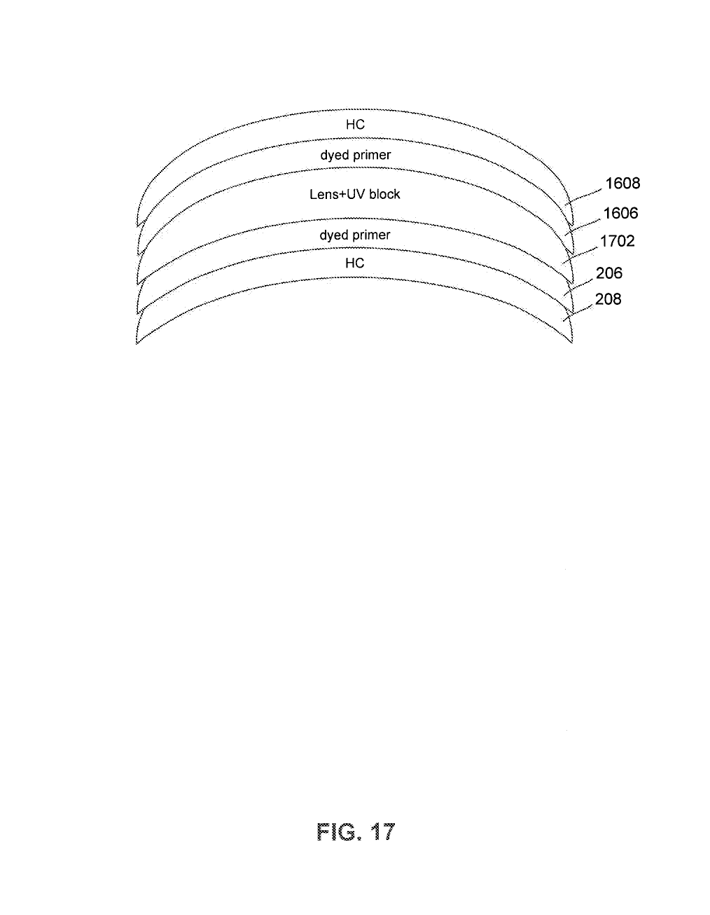

FIG. 17 illustrates an ophthalmic system according an embodiment of the invention and as described in Example 9. In FIG. 17, providing UV-blocking layers is not necessary because substrate 1702 has intrinsic UV-rejection.

Lastly, it is noted that solvent plays a particular role in the methods disclosed herein, according to embodiments of the invention. This is discussed below. Particular examples of the role of solvent are described below in the context of additional embodiments of the invention.

a) MTP dye is dissolved in cyclopentanone and added to the primer at a concentration of 1 wt % dye/primer. Then, the solution is further diluted with a fresh primer down to a concentration of 0.04 wt % dye/primer. After filtration, the solution is used for dip-coating of the lenses followed by the clear hardcoat. The final lenses show ca. 30-35% blue light blockage in the spectral range around 420 nm and YI=5.0-6.0 depending on the lens material. The haze level is between 2.0 and 3.0%.

b) MTP dye is dissolved in cyclohexanone and added to the primer at a concentration of 1 wt % dye/primer. Then, the solution is further diluted with a fresh primer down to a concentration of 0.03 wt % dye/primer. After filtration, the solution is used for dip-coating of the lenses followed by the clear hardcoat. The final lenses show ca. 30-35% blue light blockage in the spectral range around 420 nm and YI=5.0-6.0 depending on the lens material. The haze level is 0.6% or less. It is quite noticeable that the cyclohexanone is better solvent for MTP dye than cyclopentanone—the dye particles are much smaller in size in cyclohexanone and better dispersed throughout the coating. This directly reflects in much lower haze level of the final coating. Also, it is possible to achieve the same blue light blockage at a lower dye loading in a better solvent as with higher dye loading level in poor solvents (e.g. compare examples (a) and (b)).

c) MTP dye is dissolved in chloroform and added to the primer at a concentration of 1 wt % dye/primer. The solution is ultrasonicated for 2 hours at 50 C. Then, the solution is further diluted with a fresh primer down to a concentration of 0.02-0.025 wt % dye/primer and mixed well. After filtration, the solution is used for dip-coating of the lenses followed by the clear hardcoat. The final lenses show ca. 30-35% blue light blockage in the spectral range around 420 nm and YI=5.0-6.0 depending on the lens material. The chloroform seems better solvent for MTP dye compared to the examples (a) and (b) above. The same level of light blockage in the spectral range around 420 nm is achieved with lower dye concentration in chloroform.

d) MTP dye is dissolved in a solvent mixture comprising: —chlorinated solvent+ketone, or—chlorinated solvent+alcohol, or—alcohol+ketone, or—chlorinated solvent+alcohol+ketone, etc. The subsequent steps of the coating preparation process are identical as in example (c) above.

While several embodiments of the invention have been disclosed in the context of FIGS. 1-17, additional embodiments are described below. It is noted that these embodiments (as well as any of the embodiments described herein) may be combined with one another to yield additional embodiments of the invention.

In one embodiment, there is provided a method for fabricating a device that transmits light. The method comprises providing a solution containing a dye or dye mixture, and the dye or the dye mixture forms aggregates of average size less than 10 micrometers. Furthermore, the method comprises incorporating the dye or the dye mixture in the optical path of the device, and the dye or dye mixture selectively filters at least one wavelength of light within the range of 400 nm to 500 nm. Moreover, the device having the dye or dye mixture incorporated therein has an average transmission of at least 80% across the visible spectrum.

In one embodiment, the dye or dye mixture has an absorption spectrum with at least one absorption peak in the range 400 nm to 500 nm.

In one embodiment, the at least one absorption peak is in the range 400 nm to 500 nm.

In one embodiment, the at least one absorption peak has a full-width at half-max (FWHM) of less than 60 nm in the range 400 nm to 500 nm.

In one embodiment, the dye or dye mixture, when incorporated in the device's optical path, absorbs at least 5% of the at least one wavelength of light in the range 400 nm to 500 nm.

In one embodiment, the device having the dye incorporated therein has a yellowness index of 15 or less.

In one embodiment, the dye or dye mixture aggregates have an average size less than 5 micrometers.

In one embodiment, the dye or dye mixture aggregates have an average size less than 1 micrometer.

In one embodiment, providing the solution comprises ultrasonicating the solution to reduce the average size of aggregates of the dye or dye mixture contained in the solution.

In one embodiment, the ultrasonicating is performed in a controlled temperature environment.

In one embodiment, the aggregates have an average size greater than 10 micrometers prior to ultrasonicating the solution.

In one embodiment, the controlled temperature environment is set to a temperature equal or less than 50 degrees C.

In one embodiment, the incorporating comprises loading the solution in a resin to form a coating formulation.

In one embodiment, the coating formulation is subjected to further ultrasonication in a controlled temperature environment for a certain time period.

In one embodiment, the incorporating further comprises applying the coating formulation on a surface of the device.

In one embodiment, the device is an ophthalmic lens.

In one embodiment, the device is a non-ophthalmic system.

In one embodiment, the method further comprises machining a first surface of the ophthalmic lens and polishing the first surface. Furthermore, the incorporating step comprises applying a coating formulation comprising the dye or the dye mixture on the first surface to form a coating, the coating selectively inhibiting visible light in a selected range of visible wavelengths. Furthermore, the incorporating step comprises air drying or short thermal baking the coating, applying a hard scratch resistant coating on the coating,

curing the hard scratch resistant coating.

In one embodiment, the machining and the polishing provide a predetermined optical power to the ophthalmic lens.

In one embodiment, applying the coating formulation comprises determining an amount of the dye or the dye mixture, the amount corresponding to a predetermined percentage of blockage of light in the selected range.

In one embodiment, the first surface comprises a first layer which blocks ultraviolet (UV) light.

In one embodiment, a second surface of the ophthalmic lens disposed opposite to the first surface and in a plane parallel to the first surface, comprises a second layer which blocks UV light.

In one embodiment, the dye is a porphyrin or porphyrin derivative.

In one embodiment the dye is one of the group consisting of bilirubin; chlorophyll a; chlorophyll b; diprotonated-tetraphenylporphyrin; hematin; magnesium octaethylporphyrin; magnesium octaethylporphyrin (MgOEP); magnesium phthalocyanine (MgPc), PrOH; magnesium phthalocyanine (MgPc), pyridine; magnesium tetramesitylporphyrin (MgTMP); magnesium tetraphenylporphyrin (MgTPP); octaethylporphyrin; phthalocyanine (Pc); porphin; tetra-t-butylazaporphine; tetra-t-butylnaphthalocyanine; tetrakis(2,6-dichlorphenyl)porphyrin; tetrakis(o-aminophenyl)porphyrin; tetramesitylporphyrin (TMP); tetraphenylporphyrin (TPP); vitamin B12; zinc octaethylporphyrin (ZnOEP); zinc phthalocyanine (ZnPc), pyridine; zinc tetramesitylporphyrin (ZnTMP); zinc tetramesitylporphyrin radical cation; zinc tetrapheynlporphyrin (ZnTPP); perylene and derivatives thereof.

In one embodiment, the dye is tetrakis(2,6-dichlorphenyl)porphyrin (MTP).

In one embodiment, the solution includes a chlorinated solvent.

In one embodiment, the solution includes solvent having a polarity index of 3.0 or greater.

In one embodiment, the solution comprises a solvent selected from the group consisting of cyclopentanone, cyclohexanone, methyl ethyl ketone, DMSO, DMF, THF, chloroform, methylene chloride, acetonitrile, carbon tetrachloride, dichloroethane, dichloroethylene, dichloropropane, trichloroethane, trichloroethylene, tetrachloroethane, tetrachloroethylene, chlorobenzene, dichlorobenzene, and combinations thereof.

In one embodiment, the solvent of the solution is chloroform.

In one embodiment, the solvent of the solution consists essentially of chloroform.

In one embodiment, the solvent is a chlorinated solvent.

In one embodiment, the at least one wavelength of light is within the range 430 nm+/−20 nm.

In one embodiment, the at least one wavelength of light is within the range 430 nm+/−30 nm.

In one embodiment, the at least one wavelength of light is within the range 420 nm+/−20 nm.

In one embodiment, the coating is a primer coating.

In one embodiment, the method further comprises incorporating at least one of a UV-blocking component and an IR-blocking component in the optical path of the device.

In one embodiment, the method further comprises incorporating at least one of a UV-blocking component and an IR-blocking component in the optical path of the device.

In one embodiment, the device selectively filters the at least one wavelength in the range of 400 nm to 500 nm using at least one of a reflective coating and a multi-layer interference coating.

In one embodiment, the dye or dye mixture, when incorporated in the device's optical path, absorbs 5-50% of light in the range 400 nm to 500 nm.

In one embodiment, the dye or dye mixture, when incorporated in the device's optical path, absorbs 20-40% of light in the range 400 nm to 500 nm.

In one embodiment, the device blocks 5-50% of light in the range 400 nm to 500 nm.

In one embodiment, the device blocks 20-40% of light in the range 400 nm to 500 nm.

In one embodiment, the controlled temperature environment is set at a temperature equal to or less than 50 degrees C. and the time period is between 1 hour and 5 hours.

In one embodiment, the dye or dye mixture has a Soret peak within the range 400 nm to 500 nm.

In one embodiment, the at least one absorption peak has a full-width at half-max (FWHM) of less than 40 nm in the range 400 nm to 500 nm.

In one embodiment, the at least one wavelength is 430 nm.

In one embodiment, The method of claim 1, wherein the dye or dye mixture, when incorporated in the device's optical path, absorbs 5-50% of light in the range 410 nm to 450 nm.

In one embodiment, the dye or dye mixture, when incorporated in the device's optical path, absorbs 20-40% of light in the range 410 nm to 450 nm.

In one embodiment, the device blocks 5-50% of light in the range 410 nm to 450 nm.

In one embodiment, the device blocks 20-40% of light in the range 410 nm to 450 nm.

In one embodiment, the dye or dye mixture, when incorporated in the device's optical path, absorbs 5-50% of light in the range 400 nm to 460 nm.

In one embodiment, the dye or dye mixture, when incorporated in the device's optical path, absorbs 20-40% of light in the range 400 nm to 460 nm.

In one embodiment, the device blocks 5-50% of light in the range 400 nm to 460 nm.

In one embodiment, the device blocks 20-40% of light in the range 400 nm to 460 nm.

In one embodiment, the dye or dye mixture, when incorporated in the device's optical path, absorbs 5-50% of light in the range 400 nm to 440 nm.

In one embodiment, the dye or dye mixture, when incorporated in the device's optical path, absorbs 20-40% of light in the range 400 nm to 440 nm.

In one embodiment, the device blocks 5-50% of light in the range 400 nm to 440 nm.

In one embodiment, the device blocks 20-40% of light in the range 400 nm to 440 nm.

In one embodiment, the haze level of the device having incorporated therein the dye or dye mixture therein is less than 0.6%.

In one embodiment, there is provided an ophthalmic system which comprises an ophthalmic lens selected from the group consisting of a spectacle lens, contact lens, intra-ocular lens, corneal inlay, corneal onlay, corneal graft, and corneal tissue, and a selective light wavelength filter that blocks 5-50% of light having a wavelength in the range between 400-500 nm and transmits at least 80% of light across the visible spectrum. Further, the selective wavelength filter comprises a dye or a dye mixture having average aggregate size of less than 1 micrometer.

In one embodiment, the system exhibits a yellowness index of no more than 15.

In one embodiment, the system has a haze level of less than 0.6%.

In one embodiment, the range is 400-460 nm.

In one embodiment, there is provided a method comprising providing a solution containing a dye or a dye mixture, ultrasonicating the solution to reduce the average size of aggregates of the dye or dye mixture contained in the solution, and incorporating the dye or the dye mixture in the optical path of a device that transmit light.

In one embodiment, there is provided an ophthalmic system prepared by a process comprising providing a solution containing a dye or dye mixture, the dye or the dye mixture forming aggregates of average size less than 10 micrometers, incorporating the dye or the dye mixture in the optical path of the ophthalmic lens, and the dye or dye mixture selectively filters at least one wavelength of light within the range of 400 nm to 500 nm. Further, the system having the dye or dye mixture incorporated therein has an average transmission of at least 80% across the visible spectrum.

In one embodiment, the ophthalmic system comprises an ophthalmic lens, the ophthalmic lens selected from the group consisting of a spectacle lens, contact lens, intra-ocular lens, corneal inlay, corneal onlay, corneal graft, and corneal tissue. Further, the ophthalmic system comprises a selective light wavelength filter that blocks 5-50% of light having a wavelength in the range of 400-500 nm and transmits at least 80% of light across the visible spectrum, the selective wavelength filter comprising the dye or dye mixture.

In one embodiment, the system exhibits a yellowness index of no more than 15.

In one embodiment, the haze level of the ophthalmic system is less than 0.6%.

In one embodiment, the ophthalmic system comprises a selective blue light filter and a UV inhibitor, whereby the UV blocking agent is located further from the eye of the wearer than that of the selective blue light filter.

In another embodiment, the selective blue light filter is a dye.

In one embodiment, the dye is MTP.

In one embodiment, the yellowness index of the ophthalmic lens is no more than 15.0.

In one embodiment, the yellowness index of the ophthalmic lens is within the range of 1.5 to 15.0.

In one embodiment, the yellowness index of the ophthalmic lens is 10.0 or less. In another embodiment, the yellowness index is 9.0 or less. In another embodiment, the yellowness index is 8.0 or less. In another embodiment, the yellowness index is 7.0 or less. In another embodiment, the yellowness index is 6.0 or less. In another embodiment, the yellowness index is 5.0 or less. In another embodiment, the yellowness index is 4.0 or less. In another embodiment, the yellowness index is 3.0 or less.

In one embodiment, the light transmission through the ophthalmic system is 85% or greater, preferably 90% or greater.

In one embodiment, there is provided a method for making a coating which selectively filters blue light. The method comprises a first step of manufacturing the lens, a second step of adding the UV inhibitor, and a third step of applying on the lens a layer which comprises the selective filter dye package, whereby when the lens is worn by a wearer, the layer comprising the UV inhibitor is located farther away from the eye of the wearer than that of the layer comprising the selective blue light filter.

In one embodiment, the yellowness index of the lens is less than 15.0.

In one embodiment, they layer comprising the selective blue light filter is a resin layer.

In one embodiment, a solvent is used to allow the dye to be loaded within the resin layer.

In one embodiment, the solvent is one of cyclopentanone, cyclohexanone, methyl ethyl ketone, DMSO, DMF, chlorinated solvents and others, or their combination.

In one embodiment, the dye is dissolved in cyclohexanone solvent, and is loaded within the resin at very low concentrations (e.g. 0.02-0.03 wt %) and provides high blue-light-blocking levels, viz. 20-40% blue-light-blockage in the spectral range around 420 nm at low yellowness index (YI between 5 and 6) and low haze values (0.6% or less).

In one embodiment, the dye is dissolved in chloroform solvent, and it is loaded within the resin layer at very low concentrations (e.g. 0.02-0.03 wt %) and provides high blue-light-blocking levels, viz. 20-40% blue-light-blockage in the spectral range around 420 nm at low yellowness index (YI between 5 and 6).

In one embodiment, the yellowness index contribution of the selective filtering dye is within the range of 1.5 to 15.0.

In one embodiment, there is provided a non-ophthalmic system comprising a selective blue filter and a UV inhibitor.

In one embodiment of the non-ophthalmic system, the selective blue light filter is a dye.

In one embodiment of the non-ophthalmic system, the dye is MTP.

In one embodiment of the non-ophthalmic system, the yellowness index of the non-ophthalmic system is no more than 15.0.

In one embodiment the light transmission through the non-ophthalmic system is 85% or greater, preferably 90% or greater.

In one embodiment, there is provided a method for fabricating either the ophthalmic or the non-ophthalmic system wherein applying a coating comprising the selective blue light filter comprises determining an amount of the dye or the dye mixture, the amount corresponding to a predetermined percentage of blockage of light in the selected range.

While this disclosure describes many embodiments of the invention, some of which show specific layers and layer arrangements, these specific layers and layer arrangements are non-limiting. One of skill in the art will readily understand that providing selective-blue blocking layers and/or components in devices that transmit light may be achieved using the teachings disclosed herein, without specifically using the aforementioned specific layers and layer arrangements disclosed.

Further, references herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. The breadth and scope of the invention should not be limited by any of the above-described exemplary embodiments, but should be defined only in accordance with the following claims and their equivalents.

What is claimed:

1. A method for fabricating a device that transmits light, the method comprising:
providing a solution containing a dye or dye mixture, the dye or the dye mixture forming aggregates of average size less than 10 micrometers;
incorporating the dye or the dye mixture in the optical path of the device;
wherein the dye or dye mixture selectively filters at least one wavelength of light within the range of 400 nm to 500 nm; and
wherein the device having the dye or dye mixture incorporated therein has an average transmission of at least 80% across the visible spectrum.

2. The method of claim 1, wherein the dye or dye mixture has an absorption spectrum with at least one absorption peak in the range 400 nm to 500 nm.

3. The method of claim 2, wherein the at least one absorption peak has a full-width at half-max (FWHM) of less than 60 nm in the range 400 nm to 500 nm.

4. The method of claim 1, where the dye or dye mixture, when incorporated in the device's optical path, absorbs at least 5% of the at least one wavelength of light in the range 400 nm to 500 nm.

5. The method of claim 4, wherein the device having the dye incorporated therein has a yellowness index of 15 or less.

6. The method of claim 1, wherein the dye or dye mixture aggregates have an average size less than 5 micrometers.

7. The method of claim 1, wherein the dye or dye mixture aggregates have an average size less than 1 micrometer.

8. The method of claim 1, wherein providing the solution comprises ultrasonicating the solution to reduce the average size of aggregates of the dye or dye mixture contained in the solution.

9. The method of claim 8, wherein the ultrasonicating is performed in a controlled temperature environment.

10. The method of claim 1, wherein aggregates have an average size greater than 10 micrometers prior to ultrasonicating the solution.

11. The method of claim 9, wherein the controlled temperature environment is set to a temperature equal or less than 50 degrees C.

12. The method of claim 1, wherein the incorporating comprises loading the solution in a resin to form a coating formulation.

13. The method of claim 12, wherein the coating formulation is subjected to further ultrasonication in a controlled temperature environment for a certain time period.

14. The method of claim 12, wherein the incorporating further comprises applying the coating formulation on a surface of the device.

15. The method of claim 1, wherein the device is an ophthalmic lens.

16. The method of claim 1, wherein the device is a non-ophthalmic system.

17. The method of claim 15, further comprising:
machining a first surface of the ophthalmic lens;
polishing the first surface; and wherein, the incorporating comprises:
applying a coating formulation comprising the dye or the dye mixture on the first surface to form a coating, the coating selectively inhibiting visible light in a selected range of visible wavelengths;
air drying or short thermal baking the coating;
applying a hard scratch resistant coating on the coating; and
curing the hard scratch resistant coating.

18. The method of claim 17, wherein the machining and the polishing provide a predetermined optical power to the ophthalmic lens.

19. The method of claim 17, wherein applying the coating formulation comprises determining an amount of the dye or the dye mixture, the amount corresponding to a predetermined percentage of blockage of light in the selected range.

20. The method of claim 17, wherein the first surface comprises a first layer which blocks ultraviolet (UV) light.

21. The method of claim 20, wherein a second surface of the ophthalmic lens, disposed opposite to the first surface and in a plane parallel to the first surface, comprises a second layer which blocks UV light.

22. The method of claim 1, wherein the dye is a porphyrin or porphyrin derivative.

23. The method of claim 1, wherein the dye is one of the group consisting of bilirubin; chlorophyll a; chlorophyll b; diprotonated-tetraphenylporphyrin; hematin; magnesium octaethylporphyrin; magnesium octaethylporphyrin (MgOEP); magnesium phthalocyanine (MgPc), PrOH; magnesium phthalocyanine (MgPc), pyridine; magnesium tetramesitylporphyrin (MgTMP); magnesium tetraphenylporphyrin (MgTPP); octaethylporphyrin; phthalocyanine (Pc); porphin; tetra-t-butylazaporphine; tetra-t-butylnaphthalocyanine; tetrakis(2,6-dichlorphenyl)porphyrin; tetrakis(o-aminophenyl)porphyrin; tetramesitylporphyrin (TMP); tetraphenylporphyrin (TPP); vitamin B12; zinc octaethylporphyrin (ZnOEP); zinc phthalocyanine (ZnPc), pyridine; zinc tetramesitylporphyrin (ZnTMP); zinc tetramesitylporphyrin radical cation; zinc tetrapheynlporphyrin (ZnTPP); perylene and derivatives thereof.

24. The method of claim 1, wherein the dye is tetrakis(2,6-dichlorphenyl)porphyrin (MTP).

25. The method of claim 1, wherein the solution includes a chlorinated solvent.

26. The method of claim 1, wherein the solution includes solvent having a polarity index of 3.0 or greater.

27. The method of claim 1, wherein the solution comprises a solvent selected from the group consisting of cyclopentanone, cyclohexanone, methyl ethyl ketone, DMSO, DMF, THF, chloroform, methylene chloride, acetonitrile, carbon tetrachloride, dichloroethane, dichloroethylene, dichloropropane, trichloroethane, trichloroethylene, tetrachloroethane, tetrachloroethylene, chlorobenzene, dichlorobenzene, and combinations thereof.

28. The method of claim 1, wherein the solvent of the solution is chloroform.

29. The method of claim 1, wherein the solvent of the solution consists essentially of chloroform.

30. The method of claim 1, wherein the solvent is a chlorinated solvent.

31. The method of claim 1, wherein the at least one wavelength of light is within the range 430 nm+/−20 nm.

32. The method of claim 1, wherein the at least one wavelength of light is within the range 430 nm+/−30 nm.

33. The method of claim 1, wherein the at least one wavelength of light is within the range 420 nm+/−20 nm.

34. The method of claim 16, wherein the coating is a primer coating.

35. The method of claim 1, further comprising incorporating at least one of a UV-blocking component and an IR-blocking component in the optical path of the device.

36. The method of claim 16, further comprising incorporating at least one of a UV-blocking component and an IR-blocking component in the optical path of the device.

37. The method of claim 1, wherein the device selectively filters the at least one wavelength in the range of 400 nm to 500 nm using at least one of an a reflective coating and a multi-layer interference coating.

38. The method of claim 1, wherein the dye or dye mixture, when incorporated in the device's optical path, absorbs 5-50% of light in the range 400 nm to 500 nm.

39. The method of claim 39, wherein the dye or dye mixture, when incorporated in the device's optical path, absorbs 20-40% of light in the range 400 nm to 500 nm.

40. The method of claim 1, wherein the device blocks 5-50% of light in the range 400 nm to 500 nm.

41. The method of claim 41, wherein the device blocks 20-40% of light in the range 400 nm to 500 nm.

42. The method of claim 13, wherein the controlled temperature environment is set at a temperature equal to or less than 50 degrees C. and the time period is between 1 hour and 5 hours.

43. The method of claim 1, wherein the dye or dye mixture has a Soret peak within the range 400 nm to 500 nm.

44. The method of claim 3, wherein the at least one absorption peak has a full-width at half-max (FWHM) of less than 40 nm in the range 400 nm to 500 nm.

45. The method of claim 4, wherein the at least one wavelength is 430 nm.

46. The method of claim 1, wherein the dye or dye mixture, when incorporated in the device's optical path, absorbs 5-50% of light in the range 410 nm to 450 nm.

47. The method of claim 46, wherein the dye or dye mixture, when incorporated in the device's optical path, absorbs 20-40% of light in the range 410 nm to 450 nm.

48. The method of claim 1, wherein the device blocks 5-50% of light in the range 410 nm to 450 nm.

49. The method of claim 48, wherein the device blocks 20-40% of light in the range 410 nm to 450 nm.

50. The method of claim 1, wherein the dye or dye mixture, when incorporated in the device's optical path, absorbs 5-50% of light in the range 400 nm to 460 nm.

51. The method of claim 50, wherein the dye or dye mixture, when incorporated in the device's optical path, absorbs 20-40% of light in the range 400 nm to 460 nm.

52. The method of claim 1, wherein the device blocks 5-50% of light in the range 400 nm to 460 nm.

53. The method of claim 52, wherein the device blocks 20-40% of light in the range 400 nm to 460 nm.

54. The method of claim 1, wherein the dye or dye mixture, when incorporated in the device's optical path, absorbs 5-50% of light in the range 400 nm to 440 nm.

55. The method of claim 54, wherein the dye or dye mixture, when incorporated in the device's optical path, absorbs 20-40% of light in the range 400 nm to 440 nm.

56. The method of claim 1, wherein the device blocks 5-50% of light in the range 400 nm to 440 nm.

57. The method of claim 56, wherein the device blocks 20-40% of light in the range 400 nm to 440 nm.

58. The method of claim 1, wherein the haze level of the device having incorporated therein the dye or dye mixture therein is less than 0.6%.

59. An ophthalmic system comprising:
an ophthalmic lens selected from the group consisting of a spectacle lens, contact lens, intra-ocular lens, corneal inlay, corneal onlay, corneal graft, and corneal tissue, and a selective light wavelength filter that blocks 5-50% of light having a wavelength in the range between 400-500 nm and transmits at least 80% of light across the visible spectrum; and
wherein the selective wavelength filter comprises a dye or a dye mixture having average aggregate size of less than 1 micrometer.

60. The ophthalmic system of claim 59, wherein the system exhibits a yellowness index of no more than 15.

61. The ophthalmic system of claim 59, wherein the system has a haze level of less than 0.6%.

62. The ophthalmic system of claim 59, wherein the range is 400-460 nm.

63. A method, comprising:
providing a solution containing a dye or a dye mixture;
ultrasonicating the solution to reduce the average size of aggregates of the dye or dye mixture contained in the solution; and
incorporating the dye or the dye mixture in the optical path of a device that transmit light.

64. An ophthalmic system prepared by a process comprising:
providing a solution containing a dye or dye mixture, the dye or the dye mixture forming aggregates of average size less than 10 micrometers;
incorporating the dye or the dye mixture in the optical path of the ophthalmic lens;
wherein the dye or dye mixture selectively filters at least one wavelength of light within the range of 400 nm to 500 nm; and
wherein the system having the dye or dye mixture incorporated therein has an average transmission of at least 80% across the visible spectrum.

65. The ophthalmic system of claim 64, comprising:
an ophthalmic lens, the ophthalmic lens selected from the group consisting of a spectacle lens, contact lens, intra-ocular lens, corneal inlay, corneal onlay, corneal graft, and corneal tissue, and
a selective light wavelength filter that blocks 5-50% of light having a wavelength in the range of 400-500 nm and transmits at least 80% of light across the visible spectrum, the selective wavelength filter comprising the dye or dye mixture.

66. The ophthalmic system of claim 65, wherein the system exhibits a yellowness index of no more than 15.

67. The ophthalmic system of claim 66, wherein the haze level of the ophthalmic system is less than 0.6%.
